(19) United States
(12) Patent Application Publication
(10) Pub. No.: US 2003/0104458 A1
(43) Pub. Date: Jun. 5, 2003

(54) Facilitation of genome characterization by integrating renaturation kinetics with cloning and sequencing

(76) Inventors: Daniel G. Peterson, Starkville, MS (US); Andrew H. Paterson, Arnoldsville, GA (US); Susan R. Wessler, Athens, GA (US)

Correspondence Address: William L. Warren, SUTHERLAND ASBILL & BRENNAN LLP, 999 Peachtree Street, NE, Atlanta, GA, 30309-3996, US

(21) Appl. No.: 10/293,038

(22) Filed: Nov. 13, 2002

Related U.S. Application Data
(60) Provisional application No. 60/338,181, filed on Nov. 13, 2001.

(51) Int. Cl. C12Q 1/68; C12N 1/18; C12N 5/06; C12N 5/04; C12N 15/74; C12N 15/85

(52) U.S. Cl. 435/6; 435/455; 435/483; 435/325; 435/419; 435/254.2

(57) ABSTRACT

Cot-based cloning and sequencing (CBCS) is a method that permits the cloning and sequencing of an organism's sequence complexity at unprecedented efficiency. DNA renaturation kinetics (i.e., Cot) methods are used to fractionate genomic DNA into single-copy and repeat sequence components, each isolated kinetic component is used to construct a corresponding DNA library, and clones from each library are sequenced in numbers proportional to the complexity of the component from which they were derived. For some species, the number of clones that need to be sequenced in order to attain a specific level of coverage via CBCS is less than one-tenth the number required to achieve the same level of coverage using shotgun sequencing (the current means by which genomes are sequenced). In addition, the CBCS method also has advantages over other methods for sequencing low-copy or genic regions of a genome in that it secures sequences independently of their expression and/or methylation status, conditions that vary widely with different species, genes, and developmental stages.

### CROSS REFERENCE TO RELATED APPLICATIONS

The present invention claims the priority benefit of U.S. Provisional Patent Application Serial No. 60/338,181 filed Nov. 13, 2001, the entire contents of which are hereby incorporated by reference.

### ACKNOWLEDGMENT OF FEDERAL RESEARCH SUPPORT

This invention was made, at least in part, with funding from the United States Department of Agriculture (USDA-NRICGP award 99-35300). Accordingly, the United States Government has certain rights in this invention.

### BACKGROUND OF THE INVENTION

1. Field of the Invention

The field of this invention is the molecular biological analysis of complex genomes, in particular where the analysis is facilitated by the integration of renaturation kinetics with cloning and sequencing techniques, especially to improve the efficiency with which single/low copy sequences are studied.

2. Background Art

The genomes of most eukaryotes are populated by numerous repeat sequences, some of which may be repeated thousands or millions of times. Such repeats complicate all aspects of genome research and generally limit complete genome sequencing efforts to organisms with small DNA contents, unless massive efforts are undertaken, such as those seen for the sequencing of the human genome. A powerful alternative to complete genome sequencing is the isolation and sequencing of the sequence complexity of an organism. Sequence complexity is the nucleotide sequence of the combined total length in nucleotide base pairs of all of the different sequences in that genome (Britten et al., 1974 Meth. Enzymol., 29:363-405). The development of effective strategies for ‘capturing’ sequence complexity would permit the study of genetic and genomic variation in species with large DNA contents at a fraction of the cost of complete genome sequencing.

The presence of repetitive elements in eukaryotic DNA complicates many aspects of contemporary genome research, and thus substantial efforts are taken to isolate DNA regions that do not contain repetitive sequences. One means of obtaining single/low-copy sequences is to prepare cDNA libraries. However, the representation of genes in a given cDNA library is only indicative of gene expression in the source tissue(s), and gene copy number is not accurately reflected in cDNA libraries even if “normalization” techniques are employed (e.g., Ko, 1990 Nucleic Acids Res., 18:5705-5711; Soares et al., 1994 Proc. Natl. Acad. Sci. USA, 91:9228-9232; Neto et al., 1997 Gene, 186:135-142; Poustka et al., 1999 Genomics, 59:122-133).

Because repetitive DNA is often more highly methylated than low-copy DNA, some researchers have used methylation-sensitive restriction enzymes (e.g., Tanksley et al., 1988 In Chromosome Structure and Function: Impact Of New Concepts, J. P. Gustafson and R. A. Appels, Eds. (New York: Plenum Press), pp.157-173; McCouch et al., 1988 Theor. Appl. Genet., 76:815-829; Fu and Dooner, 2000 Genome Res., 10:866-873) or bacterial host strains that preferentially restrict methylated DNA (Rabinowicz et al., 1999 Nature Genet., 23:305-308) to produce genomic libraries enriched in low-copy, presumably genic, DNA. While fractionation of genomes based upon methylation status has long been used as a means of selecting for locus-specific DNA markers (McCouch, 1988 Theor. Appl. Genet., 76:815-829), it has recently been suggested as a means of capturing sequence complexity (Rabinowicz et al., 1999 Nature Genet., 23:305-308). However, the preferential exclusion of hypermethylated DNA from a genomic library could result in the loss of important and interesting genes because the pattern and significance of DNA methylation can differ markedly between species, genes within an organism, developmental stages, and different regions of the same gene (Simmen et al., 1999 Science, 283:1164-1167; Lois et al., 1990 Mol. Cell Biol., 10:16-27; Wolfl et al., 1991 Proc. Natl. Acad. Sci. USA, 88:271-275; Heslop-Harrison, 2000 Plant Cell, 12:617-635; Li et al., 1993 Nature, 366:362-365; Riesewijk et al., 1996 Genomics, 31:158-166). In addition, such methods are based on the assumption that hypermethylated sequences represent DNA that is genetically inactive and hypomethylated sequences represent low-copy DNA. Such assumptions are not necessarily true (Lois et al., 1990 Mol. Cell Biol., 10:16-27; Wolfl et al., 1991 Proc. Natl. Acad. Sci. USA, 88:271-275; Li et al., 1993 Nature, 366:362-365; Riesewijk et al., 1996 Genomics, 31:158-166; Simmen et al., 1999 Science, 283:1164-1167; Heslop-Harrison, 2000 Plant Cell, 12:617-635).

While cDNA sequencing remains an economical ‘first cut’ technique for capturing the sequence complexity of a genome, there remains a need to provide access to regulatory sequences and other genomic features that are absent from cDNA (EST) libraries, and also a need to access genes independently of their levels or tissue or organ-specific patterns of expression, increasing the likelihood of discovering key regulatory signals that are only transiently expressed. Alternative techniques to isolate DNA regions that do not contain repetitive sequences that are not subject to the constraints of currently practiced techniques are therefore required.

When a solution of denatured genomic DNA is placed in an environment conducive to renaturation, the rate at which a particular sequence reassociates is proportional to the number of times it is found in the genome. This principle forms the basis of DNA renaturation kinetics (also called “Cot analysis”), a technique by which the redundant nature of eukaryotic genomes was first demonstrated (Britten and Kohne, 1968 Science, 161:529-540). In a typical renaturation kinetics study, samples of sheared genomic DNA are heat-denatured and allowed to reassociate to different “Cot values.” A Cot value is the product of nucleotide concentration in moles per liter (C₀ or Co), the reassociation time in seconds (t), and, if applicable, a factor based upon the cation concentration of the buffer (see Britten et al. , 1974 Meth. Enzymol., 29:363-405, for review). For each sample, renatured DNA is separated from single-stranded DNA using hydroxyapatite (HAP) chromatography, and the percentage of the sample that has not reassociated (% ssDNA) is determined. The logarithm of a sample's Cot value is plotted against its corresponding % ssDNA to yield a “Cot point”, and a graph of Cot points ranging from little or no reassociation until reassociation approaches completion is called a “Cot curve” (Peterson et al., 1998 Genome, 41:346-356).

Mathematical analysis of a Cot curve permits estimation of genome size, the fraction of single-copy DNA in a genome, and the number, size, and kinetic complexity (i.e., estimated sequence complexity) of repetitive DNA classes (Peterson et al., 1998 Genome, 41:346-356). Interspecific comparison of Cot data has provided considerable insight into the structure and evolution of eukaryotic genomes (e.g., Britten and Kohne, 1968 Science, 161:529-540; Davidson et al., 1975 Chromosoma, 51:253-259; Goldberg et al., 1975 Chromosoma, 51:225-251; Galau et al., 1976 In Molecular Evolution, F. Ayala, Ed. (Sunderland, M A: Sinauer Assoc.), pp. 200-224; Hake and Walbot, 1980 Chromosoma, 79:251-270; Geever et al., 1989 Theor. Appl. Genet., 77:553-559). Based on the results of a Cot analysis, DNA renaturation kinetics and HAP chromatography can be used to fractionate genomic DNA into complexity-based components (Britten et al., 1974 Meth. Enzymol., 29:363-405; Britten and Kohne, 1968 Science, 161:529-540; Peterson et al., 1998 Genome, 41:346-356; Goldberg, 1978 Biochem. Genet., 16:45-68).

Cot analysis was virtually abandoned with the advent of molecular cloning, and consequently isolated Cot components previously have not been used to construct genomic DNA libraries that allow for the determination of sequence complexity. While a fold-back Cot component from maize was cloned and analyzed by Southern blot (McElfresh and Strommer, 1986 Maize Newsletter, 60:17), this study did not provide a method for analyzing the sequence complexity of an organism, i.e. information about the kinetic complexity of a component was not used to determine the number of clones that needed to be studied in order to provide a substantial representation of the component, and consequently too few clones were studied. Similarly, a combination of single/low copy and moderately repetitive DNA components was cloned, and 48 clones were sequenced in order to analyze the microsatellites of a conifer genome (Elsik and Williams, 2000 Mol. Gen. Genet., 264: 47-55). The study by Elsik and Williams also does not link sequencing depth of a Cot fraction to the sequence complexity of an organism, i.e. like McElfresh and Strommer, information about the kinetic complexity of the component was not linked to the number of clones sequenced, and consequently the experiment failed to provide meaningful coverage of the sequence complexity of the component. Cot/hydroxyapatite techniques have also been used to construct normalized cDNA libraries (Davidson, et al., 1973 J. Mol. Biol., 77: 1-23; Ko, 1990 Nucleic Acids Res., 18: 5705-5711), isolate repetitive genomic DNA for use in chromosomal in situ suppression hybridization (Landegent, et al., 1987 Hum. Genet., 77: 366-370), to clone DNA regions associated with known chromosomal deletions/additions using the phenol emulsion reassociation technique (Kunkel, et al., 1985 Proc. Natl. Acad. Sci. USA, 82: 4778-4782; Clarke, et al., 1992 Nucleic Acids Res. 20: 1289-1292), and to characterize several highly repetitive elements from the ginseng genome (Ho & Leung, 2002 Mol. Genet. Genomics, 266: 951-961). None of these techniques provides a method for analyzing the sequence complexity of an organism.

Because of the difficulty in obtaining relatively rare single/low copy sequence information from organisms with highly repetitive genomes, there is a need in the art for techniques to improve the efficiency and economics of analyzing single/low copy sequence information from complex genomes.

### SUMMARY OF THE INVENTION

It is an object of the present invention to overcome, or at least alleviate, one or more of the difficulties or deficiencies associated with the prior art. In that regard, the present invention provides a method that improves the efficiency of cloning and sequencing genetic information, especially single/low-copy and genic sequences, from genomes that are characterized by a significant proportion of repetitive DNA. The method involves the integration of Cot based DNA fractionation and high-throughput DNA sequencing techniques. The invention, hereafter referred to as “Cot-based cloning and sequencing” (CBCS), can be used to capture most (if not all) of the sequence complexity of a genome in a manner independent of sequence methylation and gene expression patterns.

The invention provided herein encompasses methods for the cloning and analysis of the genomic DNA of an organism. Preferably, a Cot library of an organism is produced by cloning renaturation based fractionated genomic DNA into a suitable vector. In other embodiments of the invention, the sequence complexity of the organism is determined by sequencing Cot clones from a Cot library to a depth determined based on the kinetic complexity of the isolated Cot component from which the Cot library is prepared. The invention also contemplates that the methods of the invention can be used to clone and sequence the single/low copy DNA of an organism to an appropriate level determined by its kinetic complexity, by preparing and sequencing a Cot library prepared from the single/low copy Cot component isolated using renaturation kinetics based fractionation. Similarly, the invention contemplates the cloning and sequencing of the moderately repetitive and the highly repetitive DNA of an organism to appropriate levels determined by their kinetic complexity, by preparing and sequencing Cot libraries prepared from the moderately repetitive and highly repetitive Cot components isolated using renaturation kinetics based fractionation. The invention further encompasses a kit for use in nucleic acid sequencing, comprising a Cot library prepared from an isolated Cot component selected from a group consisting of a single/low copy Cot component, a moderately repetitive Cot component, and a highly repetitive Cot component.

Renaturation based kinetic fractionation is typically performed using methods including but not limited to hydroxyapatite (HAP) chromatography or preferential nuclease digestion of single-stranded DNA. Each isolated component consists of a group of sequences that possess similar kinetic properties and without wishing to be bound by theory, sequence complexities. For example, a genome can be fractionated into highly repetitive (HR), moderately repetitive (MR), and single/low-copy (SL) sequence components. Each isolated component (or subcomponent) is ligated into an appropriate vector and used to transform suitable host cells. The clones produced from a specific component or subcomponent are referred to as “Cot libraries.” Clones from Cot libraries are sequenced, preferably using high-throughput methods. To obtain comparable levels of sequence complexity coverage for different Cot libraries, clones from each Cot library can be sequenced in proportion to the kinetic complexity of the component from which they were derived. Preferably, more than 0.5% of the genome is sequenced using the above methods.

### BRIEF DESCRIPTION OF THE DRAWINGS

FIG. 1 provides an overview of steps involved in a Cot analysis, using a plant as an example. Plant leaves are homogenized in a blender, and nuclei are purified by centrifugation. DNA is isolated from purified nuclei, the DNA is sheared, and fragment size is checked by agarose gel electrophoresis. The sheared DNA is precipitated, and dissolved in sodium phosphate buffer (SPB) to produce solutions of known concentrations. The DNA solutions are distributed into glass microcapillary tubes or glass ampoules, and the ends of the tubes/ampoules are sealed. Each of the tubes are placed in boiling water to denature DNA duplexes, and renaturation is allowed to occur until each sample reaches a specific Cot value. Once the sample has reached the desired Cot value, it is loaded onto a hydroxyapatite (HAP) column. SBP is used to elute the ssDNA and the dsDNA separately. The ssDNA and the dsDNA samples are denatured, and the A260 values are determined. The logarithms of Cot values ranging from essentially no renaturation to nearly complete renaturation are plotted against corresponding % ssDNA values to yield Cot points. A graph of Cot points ranging from little or no reassociation until reassociation approaches completion is called a Cot curve.

FIG. 2 shows the results of sorghum Cot analysis. The top curve (open circles) illustrates complete Cot curve, data analysis, and component isolation. A least-squares curve (thick line) was fit through the data points (open circles). The curve consists of highly repetitive (HR), moderately repetitive (MR), and single/low-copy (SL) components characterized by fast, intermediate, and slow reassociation, respectively. For each component, the following values have been placed to the right of the component's general location: Fraction=the proportion of the genome found in that component, Complexity=the length in nucleotide pairs of the longest non-repeating sequence, k=the observed reassociation rate in M⁻¹·s⁻¹, and Cot½=the value on the abscissa of the complete Cot curve at which half the DNA in the component has reassociated. Diamonds mark the positions on the complete Cot curve of the Cot½ values for HR, MR, and SL components. The curves at the lower portion of the graph show the predicted individual renaturation profiles of the HR component, MR component, and SL component.

FIG. 3 exemplifies the relationship between sequence complexity and sequencing. (a) The elements constituting a hypothetical eukaryotic genome. (b) Though repetitive sequences account for the majority of DNA, they contribute very little to sequence complexity. (c) The net gain in novel sequence information is slow and costly if clones are selected from an unbiased genomic library (shotgun approach). (d) CBCS permits the HR, MR, and SL components of the genome to be separately isolated and cloned. Since almost all of the sequence complexity is contained within the SLCot library, most sequencing resources can be devoted to sequencing SLCot clones.

FIG. 4 compares the efficiency for CBCS versus shotgun sequencing. For each species, the number of Cot clones that would need to be sequenced to attain a specific level of sequence complexity coverage has been divided by the number of randomly-selected clones (i.e., “shotgun clones”) that would have to be sequenced in order to attain the same level of coverage. The “Cot/shotgun” ratio for each species was determined using the following formula: Ratio=(γ_{CR}+γ_{SL}+F)÷G where γ_{CR}=the sum of the kinetic complexities of all repetitive components in the genome, γ_{SL}=the kinetic complexity for the single-copy or single/low-copy component, F=the base pair content of the foldback fraction, and G=1C genome size in bp. Resulting values have been plotted against genome size. Cot and genome size information used in generating this figure can be found at www.plantgenome.uga.edu/CBCS or at www.msstate.edu/research/mgel/cbcs/cbcs.htm.

FIG. 5 provides an overview of the steps involved in cloning HR, MR, and SL Cot components. Single-stranded DNA (ssDNA) and double-stranded (dsDNA) were separated using hydroxyapatite (HAP) chromatography. To attain the equivalent of a specific Cot value when starting with an isolated Cot fraction, the desired Cot value should be multiplied by the fraction of the genome remaining single-stranded at the Cot value of the starting material. This principle was employed once in the isolation of the MR component and once in the SL component isolation: From the Cot curve, 0.67 of the genome is single-stranded at a Cot of 0.94161. To achieve renaturation equivalent to Cot 67.8 with whole genomic DNA, the Cot>0.94161 DNA was renatured to a Cot of (67.8×0.67=) 45.4. From the Cot curve, 0.28 of the genome is single-stranded at a Cot of 94.161. To achieve renaturation equivalent to Cot 10,000 with whole genomic DNA, the Cot>94.161 DNA was renatured to a Cot of (10,000×0.28=) 2800.

FIG. 6 illustrates the scheme for classification of Cot clones based on sequence analysis. For comparative purposes, each of the sequenced Cot clones meeting the minimum sequence quality requirements (see Examples) was assigned to a single descriptive “BLAST category” based upon its significant database hits according to the scheme shown.

FIG. 7 displays the composition of different Cot fractions. Black bars represent HRCot clones, white bars represent MRCot clones, and diagonally-striped bars represent SLCot clones. The BLAST group “Dispersed repeat sequences” is composed of the “Retroelement,” “MITE,” and “Other dispersed repeat” BLAST categories (see FIG. 4 and Table 2).

FIGS. 8A-8E illustrate the analysis of Retrosor-6. FIG. 8A shows the structure of Retrosor-6 and the distribution of the 41 Cot clones with primary homology to Retrosor-6. Retroelement features of Retrosor-6 include (α) duplicated target site sequences flanking both ends of the sequence, (β) long terminal repeats (LTRs) (bases 1-1279 and 6098-7377) with the canonical LTR start/end nucleotides 5′-TG . . . CA-3′, (χ) a primer binding site complementary to the plant tRNA for asparagine (bases 1286-1301), (δ) an internal sequence region with homology to ORFI of the Arabidopsis gypsy-type retroelement Athila, and (ε) a polypurine tract (bases 6083-6095) (see Murphy et al., 1995 In Virus Taxonomy: Classification and Nomenclature of Viruses. F. A. Murphy, C. M. Fauquet, D. H. L. Bishop, S. A. Ghabrial, A. W. Jarvis, G. P. Martelli, M. A. Mayo and M. D. Summers, Eds. (New York: Springer-Verlag), pp. 193-204 for review of retroelement structure). A scale showing distance in base pairs has been positioned underneath the diagram of the retroelement. For each Cot clone recognizing Retrosor-6, a thin line has been placed above the retroelement marking the relative position(s) and length of the sequence shared by that clone and Retrosor-6. Because the LTRs have almost identical sequences (99.5% sequence identity), all of the clones with homology to one LTR have a similar/identical degree of homology to the other LTR. For these clones, lines have been positioned above both LTRs. FIG. 8B shows hybridization of a Retrosor-6 probe (diamond-headed arrow in FIG. 8A) to a Southern blot. The labels at the head of each lane indicate the source of DNA in that lane and the restriction enzyme with which the DNA was digested. Specifically, b=S. bicolor, p=S. propinquum, E1=EcoR1, H3=HindIII, E5=EcoRV, and X1=XbaI. The two species show essentially identical hybridization patterns and intensities. FIG. 8C is a S. bicolor grid probed with a sequence from the Retrosor-6 LTR (chevron-headed arrow in 8A). FIG. 8D is a grid identical to that in FIG. 8C probed with a Retrosor-6 partial internal sequence (triangular-head arrow in FIG. 8A). The hybridization patterns observed for grids probed with the internal Retrosor-6 sequence are virtually identical to those produced by the LTR sequence probe for both S. bicolor (FIGS. 8C and 8D) and S. propinquum (data not shown). FIG. 8E is a photograph of a section of the S. propinquum BAC grid probed with part of the LTR sequence of Retrosor-6. The number of copies of Retrosor-6 in the S. propinquum and S. bicolor genomes were estimated as described in Table 3 and the Examples herein. The region on the grid used as “background” is enclosed within a circle. Examples of clones showing relatively intense hybridization signals are marked by arrows (triangular-heads) while clones with relatively weak but interpretable hybridization signals are marked by arrowheads (chevrons).

### DETAILED DESCRIPTION OF THE INVENTION

Applicants have demonstrated that Cot-based cloning and sequencing (CBCS) is a useful and powerful means to isolate and clone genomic sequences based upon their relative iteration, and that it allows efficient discovery of new DNA sequences in a manner independent of expression and/or methylation patterns. Additionally, high throughput sequencing of Cot libraries to levels that are determined based on their kinetic complexities represents a means by which the sequence complexity of relatively large genomes can be captured and analyzed at a fraction of the cost of shotgun cloning and sequencing.

The invention provided herein encompasses methods for the analysis of the genomic DNA of an organism. Preferably, a Cot library of an organism is produced by cloning renaturation based fractionated genomic DNA into a suitable vector. In other embodiments of the invention, the sequence complexity of the organism is determined by sequencing a Cot clone from a Cot library to a depth determined based on the kinetic complexity of the isolated Cot component from which the Cot library is prepared. The invention also contemplates that the methods can be used to clone and sequence the single/low copy DNA of an organism, by preparing and sequencing a Cot library from the single/low copy Cot component isolated using renaturation kinetics based fractionation. The invention further encompasses a kit for use in nucleic acid sequencing, comprising a Cot library prepared from an isolated Cot component selected from a group consisting of a single/low copy Cot component, a moderately repetitive Cot component, and a highly repetitive Cot component.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention particularly provides a method of producing a genomic DNA library of an organism, comprising: a) performing renaturation kinetics based fractionation of the genomic DNA of the organism; b) isolating more than one Cot component from the fractionated genomic DNA; and c) preparing more than one Cot library from the more than one isolated Cot component, to thereby produce a genomic DNA library of an organism. In one preferred embodiment, preparing the Cot library comprises a) ligating the isolated Cot component from the fractionated genomic DNA into a suitable vector; and b) transforming a host cell with the vector comprising the isolated Cot component, to thereby prepare the Cot library. In another preferred embodiment, the isolated Cot component is selected from the group consisting of a fold-back DNA fraction, a highly repetitive DNA component, a moderately repetitive DNA component, and a single/low copy DNA component.

The present invention further encompasses a method of determining the sequence complexity of the genomic DNA of an organism, comprising a) performing renaturation kinetics based fractionation of the genomic DNA of the organism; b) isolating a Cot component from the fractionated genomic DNA; c) preparing a Cot library from the isolated Cot component wherein the Cot library comprises a Cot clone; and d) sequencing a Cot clone from the Cot library to a depth determined based on the kinetic complexity of the isolated Cot component from which the Cot library is prepared, thereby determining the sequence complexity of the genomic DNA of the organism. In a preferred embodiment, the above method is utilized to sequence more than approximately 0.5% of the genomic DNA of the organism. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% of the genomic DNA of the organism.

The methods contemplated by the invention can comprise preparing a Cot library. In a preferred embodiment, preparing the Cot library comprises a) ligating the isolated Cot component from the fractionated genomic DNA into a suitable vector; and b) transforming a host cell with the vector comprising the isolated Cot component, to thereby prepare the Cot library. In another preferred embodiment, the isolated Cot component is selected from the group consisting of a fold-back DNA fraction, a highly repetitive DNA component, a moderately repetitive DNA component, and a single/low copy DNA component.

The present invention further encompasses a method of cloning the single/low copy genomic DNA of an organism, comprising a) performing renaturation kinetics based fractionation of the genomic DNA of the organism; b) isolating a single/low copy Cot component from the fractionated genomic DNA; and c) preparing a Cot library from the single/low copy Cot component wherein the Cot library comprises a Cot clone, thereby cloning the single/low copy DNA of the organism. In a preferred embodiment, the single/low copy Cot component comprises a DNA sequence that is present from approximately 1 to 10 copies per haploid genome. In other embodiments, the DNA sequence is present in fewer than approximately 9 copies, 8 copies, 7 copies, 6 copies, 5 copies, 4 copies, 3 copies, or 2 copies per haploid genome. In a further preferred embodiment, the method of the invention comprises sequencing a Cot clone from the single/low copy DNA Cot library to a depth determined based on the kinetic complexity or other estimate of iteration frequency of individual elements in the component. In a preferred embodiment, the above method is utilized to sequence more than approximately 0.5% of the genomic DNA of the organism. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% of the genomic DNA of the organism.

The present invention further encompasses a method of cloning the moderately repetitive genomic DNA of an organism, comprising a) performing renaturation kinetics based fractionation of the genomic DNA of the organism; b) isolating a moderately repetitive Cot component from the fractionated genomic DNA; and c) preparing a Cot library from the moderately repetitive Cot component wherein the Cot library comprises a Cot clone, thereby cloning the moderately repetitive DNA of the organism. In a preferred embodiment, the moderately repetitive Cot component comprises a DNA sequence that is present at more than approximately 10 copies per haploid genome. In other embodiments, the DNA sequence of the moderately repetitive Cot component is present in between 11 and 4999 copies per haploid genome. In a further preferred embodiment, the method of the invention comprises sequencing a Cot clone from the moderately repetitive DNA Cot library to a depth determined based on the kinetic complexity of the isolated Cot component. In a preferred embodiment, the above method is utilized to sequence more than approximately 0.5% of the genomic DNA of the organism. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% of the genomic DNA of the organism.

The present invention further encompasses a method of cloning the highly repetitive genomic DNA of an organism, comprising a) performing renaturation kinetics based fractionation of the genomic DNA of the organism; b) isolating a highly repetitive Cot component from the fractionated genomic DNA; and c) preparing a Cot library from the highly repetitive Cot component wherein the Cot library comprises a Cot clone, thereby cloning the highly repetitive DNA of the organism. In a preferred embodiment, the highly repetitive Cot component comprises a DNA sequence that is present at more than approximately 5000 copies per haploid genome. In a further preferred embodiment, the method of the invention comprises sequencing a Cot clone from the highly repetitive DNA Cot library to a depth determined based on the kinetic complexity of the Cot component. In a preferred embodiment, the above method is utilized to sequence more than approximately 0.5% of the genomic DNA of the organism. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% of the genomic DNA of the organism.

The present invention further encompasses a method of determining the sequence of the genomic DNA of an organism, comprising: a) performing renaturation kinetics based fractionation of the genomic DNA of the organism; b) isolating a Cot component from the fractionated genomic DNA; and c) sequencing the Cot component to a depth determined based on the kinetic complexity of the isolated Cot component, thereby determining the sequence of the genomic DNA of an organism. In a preferred embodiment, a Cot library is prepared from the isolated Cot component prior to sequencing the Cot component. In another preferred embodiment, the Cot component is sequenced without first preparing a Cot library. In a preferred embodiment, the above method is utilized to sequence more than approximately 0.5% of the genomic DNA of the organism. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% of the genomic DNA of the organism.

In one embodiment of the above methods, performing renaturation kinetics based fractionation of the genomic DNA comprises the preparation of a Cot curve. In a further preferred embodiment, the Cot curve comprises one Cot component. In another preferred embodiment, the Cot curve comprises more than one Cot component. In another preferred embodiment, the Cot curve comprises two Cot components. In another preferred embodiment, the Cot curve comprises three Cot components. The Cot components are preferably distinguished using accepted mathematical standards, preferably as set out in Pearson et al., 1977 Nucleic Acids Res., 4:1727-1737.

The present invention further encompasses a kit for use in nucleic acid sequencing. In a preferred embodiment, the kit for use in nucleic acid sequencing comprises a Cot library, wherein the Cot library is prepared from an isolated Cot component of the genomic DNA of an organism, wherein the Cot component is isolated by renaturation kinetics based fractionation of the genomic DNA. In a preferred embodiment, the isolated Cot component is selected from the group consisting of a single/low copy Cot component, a moderately repetitive Cot component, and a highly repetitive Cot component. In a preferred embodiment, the single/low copy Cot component comprises a DNA sequence that is present from approximately 1 to 10 copies per haploid genome. In other embodiments, the DNA sequence is present in fewer than approximately 9 copies, 8 copies, 7 copies, 6 copies, 5 copies, 4 copies, 3 copies, or 2 copies per haploid genome. In another preferred embodiment, the moderately repetitive Cot component comprises a DNA sequence that is present at more than approximately 10 copies per haploid genome. In other embodiments, the DNA sequence of the moderately repetitive Cot component is present in between 11 and 4999 copies per haploid genome. In a further preferred embodiment, the highly repetitive Cot component comprises a DNA sequence that is present in more than approximately 5000 copies per haploid genome.

The present invention provides for methods that allow the production of a genomic DNA library of an organism, the determination of the sequence complexity of an organism by sequencing to a depth determined based on the kinetic complexity or other estimate of iteration frequency of individual elements in the component, the determination of the sequence of the single/low copy DNA of an organism, the determination of the sequence of the moderately repetitive DNA of an organism, the determination of the sequence of the highly repetitive DNA of an organism, the determination of the sequence of the genomic DNA of an organism, and the invention further provides for kits for use in sequencing the nucleic acid of an organism.

In preferred embodiments of the present invention, the organism is a eukaryote. Non-limiting examples of the eukaryotic cells of the present invention include cells from animals, plants, fungi, protists, and other microorganisms. In some embodiments, the cells are part of a multicellular organism, e.g., a plant or animal. In one embodiment, the organism is an animal, wherein the animal is selected from the group consisting of a vertebrate and an invertebrate. In one embodiment, the eukaryotic cell is a mammalian cell. In another embodiment, the eukaryotic cell is a plant cell. Among the plant targets of particular interest are monocots, including, for example, rice, corn, wheat, rye, barley, banana, palm, lily, orchid, and sedge plants. Dicots are also suitable targets, including, for example, tobacco, apple, potato, beet, carrot, willow, elm, maple, rose, buttercup, petunia, phlox, violet, sunflower, soybean, canola, alfalfa, clover, bean, peanut, cotton and tomato. The plant target can be a conifer, a bryophyte, a fern, a hornwort, a liverwort, a horsetail, a whisk-fern, a cycad, a gingko, and a gnetophyte. Gymnosperms including, for example, pine, cedar, spruce, and hemlock can be used according to the present invention. Finally, basal angiosperms including, for example, tuliptree, eucalyptus, magnolia, and avocado can be used according to the present invention.

In preferred embodiments of the current invention, renaturation kinetics based fractionation is performed on genomic DNA fragmented by enzyme digestion, sonication, NaOH treatment, hydrodynamic shearing, or mechanical shearing. Preferably, when using enzyme digestion to fragment the genomic DNA, the enzyme is a restriction enzyme, or is a dsDNAse. Preferably, renaturation kinetics based fractionation is performed on genomic DNA having a length of between approximately 10 base pairs to approximately 10,000 base pairs, more preferably the genomic DNA has a length of between approximately 100 base pairs to approximately 1000 base pairs, and most preferably the genomic DNA has a length of between approximately 300 base pairs to approximately 600 base pairs.

The present invention contemplates that the isolated Cot component may be fractionated into more than one subcomponent prior to preparing the Cot library. In one embodiment, the Cot library is prepared from ssDNA. In another embodiment, the Cot library is prepared from dsDNA.

To test the efficacy of preparing genomic libraries from fractionated kinetic components, Cot/HAP techniques were used to isolate highly repetitive (HR), moderately repetitive (MR), and single/low-copy (SL) components from Sorghum bicolor genomic DNA, and these components were cloned to produce HRCot, MRCot, and SLCot libraries, respectively. Sequence and filter hybridization analyses indicate that the sorghum Cot libraries are representative of the components from which they were derived.

The results of a Cot analysis (or Cot parameters estimated from other genome data) can be used to guide the fractionation of a genome into kinetic components in a manner independent of sequence expression or methylation. Such fractionation can be performed by coupling Cot principles with HAP chromatography or any other technique that can separate ssDNA and dsDNA from partially renatured DNA samples.

Cot-based DNA cloning (CBDC) is one invention provided in the present invention. CBDC consists of the following steps: (1) A Cot analysis is performed for a species of interest, data is obtained from a previously published Cot analysis, or an educated guess is made regarding the Cot values that flank a DNA region(s) containing sequences of a desired sequence complexity. (2) Based on the Cot analysis data or a hypothesis regarding a suitable Cot range(s) of interest, renaturation kinetics based fractionation techniques, which include but are not limited to Cot techniques, HAP chromatography, preferential nuclease digestion of single-stranded DNA, and other fractionation methods by which ssDNA and dsDNA are separated from mixtures of partially-renatured DNA, are employed to obtain one or more isolated Cot components. If desired, an isolated Cot component can be further fractionated into more than one subcomponent. Foldback DNA can also be cloned to produce a foldback Cot library. (3) An isolated Cot component or subcomponent is ligated into an appropriate vector and used to transform an appropriate host cell line to produce a Cot library. The isolated Cot component that is cloned can comprise ssDNA or can comprise dsDNA. Preferably, a genomic DNA library of an organism is produced by preparing more than one Cot library from more than one isolated Cot component.

Cot-based Cloning and Sequencing (CBCS) is another invention provided herein. CBCS consists of the following steps: (1) performing CBDC on the genomic DNA of an organism; and (2) sequencing clones from more than one Cot library to a depth determined based on the kinetic complexity of the isolated Cot component. In a preferred embodiment, the sequencing of clones is performed using automated, high-throughput sequencing techniques. For a particular Cot library, the number of clones that need to be sequenced in order to attain a specific level of sequence complexity coverage is directly proportional to the kinetic complexity or other estimate of iteration frequency of individual elements in the Cot component from which the library was derived. Consequently, CBCS allows the development of species-specific genome sequencing strategies. For species with considerable quantities of repetitive DNA, CBCS will allow the capture of the genome sequence complexity at a fraction of the cost and effort to obtain comparable coverage using shotgun sequencing.

The present invention contemplates that if the DNA fragments in an isolated Cot component are of a length that is optimal for automated sequencing (about 500-1000 bp as of the year 2001), the fragments are cloned individually into a vector using standard techniques. If the DNA fragments are relatively short, fragments can be joined together using DNA linkers with highly recognizable sequences. In the latter situation, ligation reaction conditions are controlled so that resulting concatemers have mean lengths within the optimal sequencing size range. The generation and cloning of concatemers described above is similar to the Serial Analysis of Gene Expression technique (SAGE; Velculescu et al., 2000 Trends Genet., 16: 423-425).

In one embodiment of the present invention, the Cot-based cloning and sequencing of the present invention is combined with concatemer production. Relatively short dsDNA fragments are ligated with linkers of known nucleotide sequence between them, with about 2-5 fragments joined per recombinant DNA molecule. Preferably, the dsDNA fragments are approximately 50 bp to approximately 500 bp, and more preferably the fragments are approximately 350 bp. The cloning and sequencing of these concatemeric recombinants permits a reduction in the number of clones to be analyzed, and the known linker punctuates and separates the DNA sequences that are not, in nature, contiguous.

The CBCS approach does not provide key information such as data on point mutations in individual members of repetitive DNA families that are important for disambiguation and assembly of complete genomic sequences. However, coupling resequencing techniques (e.g., Nickerson et al., 1997 Nucl. Acids Res., 25: 2745-2751; Hacia, 1999 Nat. Genet., 21: 42-47; Kurg et al., 2000 Genet. Test. 4: 1-7; Xiao and Oefner, 2001 Hum. Nut., 17: 439-474) with the CBCS techniques described herein remedies the limitation, allowing CBCS to be used as a tool in complete genome sequencing efforts. Regardless, the improvement in the ability to capture the sequence complexity of a higher organism with far less investment than is required by shotgun sequencing greatly accelerates the timetable for genome-wide study of many of the world's biota, especially crops of agricultural and economic importance.

The examples described herein demonstrate that renaturation kinetics and molecular biology can be advantageously united to construct repetition-based genomic libraries from isolated Cot components. While the goals of the initial study were to investigate the feasibility/usefulness of Cot-cloning and further characterize the genome of sorghum, Cot clones can be employed in other ways and applied to genomic analysis of other organisms. Additionally, many of the experimental parameters utilized in this project can be altered to meet different research needs, as readily apparent to one of ordinary skill in the art.

In species where methylation is known to be associated with repetitive DNA (e.g., Rabinowicz et al., 1999 Nature Genet., 23:305-308), cloning of isolated single/low copy sequences into mcrBC⁺/mcrA⁺/mrr⁺ bacterial strains should substantially decrease contamination of the resulting library with repeat sequences. To isolate highly repetitive sequences, the opposite strategy can be employed, for example, HRCot DNA can be cloned into bacterial strains that actively restrict unmethylated DNA. Additionally, EST/cDNA and genomic libraries can be screened with isolated Cot fractions to identify populations of clones containing probable single/low copy and/or repetitive sequences, and renaturation kinetics can be used to further purify/characterize isolated Cot components to increase the resolution of Cot analysis, and subsequently increase the resolution of Cot cloning. In one embodiment, minicot analysis can be used to further purify and/or characterize the isolated Cot components (Britten et al., 1974 Meth. Enzymol., 29:363-405; Goldberg, 1978 Biochem. Genet., 16:45-68; Kiper and Herzfeld, 1978 Chromosoma, 65:335-351).

In the research described herein, only double-stranded DNA resulting from reassociation of denatured DNA was used in preparing Cot libraries. However, HAP-fractionated single-stranded DNA can be used in Cot cloning as well. In this regard, single-stranded Cot DNA (foldback sequences) have been obtained, complementary strands were generated via the random primer method (Mackey et al., 1995 FOCUS, 17:87-89), and TA-cloning techniques (Kawata et al., 1998 Curr. Microbiol., 37:289-291) were used to produce ssDNA-derived Cot clones. The use of ssDNA fractions in cloning is advantageous in instances where the quantity of genomic DNA is limited.

Many of the Cot analysis/HAP fractionation procedures can readily be automated and standardized, using currently available technology. Additional parameters that can be adapted to attain specific research goals include manipulation of renaturation stringency and/or DNA fragment length (e.g., Britten et al., 1974 Meth. Enzymol., 29:363-405; Goldberg et al., 1975 Chromosoma, 51:225-251; Walbot and Dure, 1976J. Mol. Biol., 101:503-536; Zimmerman and Goldberg, 1977 Chromosoma, 59:227-252), incorporation of S1 nuclease-digestion into the Cot analysis procedure (e.g., Smith et al., 1975 Proc. Natl. Acad. Sci. USA, 72:4805-4809; Goldberg, 1978 Biochem. Genet., 16:45-68; Kiper and Herzfeld, 1978 Chromosoma, 65:335-351; Hake and Walbot, 1980 Chromosoma, 79:251-270), and mixing of DNA/RNA from two or more different species (see Galau et al., 1976 In Molecular Evolution, F. Ayala, Ed. (Sunderland, MA: Sinauer Assoc.), pp. 200-224).

The Cot-based DNA cloning and sequencing methods of the present invention can be used in the following applications which include, but are not limited to, the capture of the sequence complexity of eukaryotic genomes (including those of large genome species); a tool in the sequencing of entire genomes; the preferential isolation and sequencing of the unique elements and genes of a genome; the preferential isolation and sequencing of the repetitive elements within a genome; the discovery of rarely expressed genes and/or genes expressed in short developmental timeframes; the discovery of genes located within heterochromatin and other highly-methylated chromosomal regions; the discovery and characterization of transposons, miniature inverted-repeat transposable elements (MITEs), and other repetitive elements that play important roles in the evolution of eukaryotic genomes and are useful as DNA markers or mutagenic agents for identifying the functions of genes by ‘knockout’ methods; the discovery of simple-sequence repeats (SSRs) or other DNA sequences specifically in low-copy regions of the genome that could be used as DNA markers in molecular mapping; and in the discovery of regulatory genomic sequences (promoters and enhancers) that determine the level and timing of expression of a gene, but are absent from cDNA libraries.

The following definitions are provided to clarify terms used in the present application.

As used herein a “gene” is the fundamental physical and functional unit of heredity. In biochemical terms, a gene is an ordered sequence of nucleotides located in a particular position on a particular chromosome that encodes a specific functional product (i.e., a protein or RNA molecule). As used in this document, a gene is composed not only of coding sequences but adjacent DNA regions involved in control of the transcription of the coding sequences (e.g., promoters, enhancers) and introns.

As used herein, a eukaryote “chromosome” is a linear double-stranded DNA molecule associated with numerous packaging proteins (e.g., histones) and regulatory proteins. Chromosomes are contained within the nucleus. Some of the DNA sequences in a chromosome are genes and some serve structural functions (e.g., telomeres and centromeres). Other chromosomal sequences serve no known genetic or structural function.

As used herein, “single-copy DNA” refers to those sequences found only once in a genome (1C).

As used herein, “repetitive DNA” refers to those sequences that are found in the genomes of eukaryotes in more than approximately 10 copies per 1C genome. Many repetitive sequences are found in thousands or millions of copies per genome. The repetitive sequences are either identical or similar in sequence. Non-limiting examples of repetitive DNA sequences include B2 sequences in mouse and Alu sequences in humans. Repetitive DNA sequences may serve structural purposes (e.g., centromeres and telomeres), but generally they serve no known function. Some repetitive sequences are derived from multiple insertions of viral DNA molecules into a host genome. Other repeat sequences appear to be very simple in nature and have been replicated through a variety of mechanisms. The genomes of many eukaryotes contain more repetitive DNA than single- or low-copy DNA.

As used herein, “chromosome sequencing” is the elucidation of the base pair sequence of one (or both) strands of a complete chromosomal DNA molecule from one end to the other. Sequencing entire eukaryotic chromosomes is extremely complex, especially in species that possess numerous repetitive DNA sequences.

As used herein, “genome sequencing” is the sequencing of all the chromosomes that comprise a genome.

As used herein, “sequencing” refers to any method known now or in the future for determining the order in which the nucleic acid bases are arranged within a length of DNA. For a general description of DNA sequencing, and various DNA sequencing techniques, reference is made generally to F. M. Ausubel et al., Eds., Current Protocols in Molecular Biology, Current Protocols, a joint venture between Greene Publishing Associates, Inc. and John Wiley & Sons, Inc., (Supplement 37, current through 1997), and in particular to Chapter 7, the entirety of which is incorporated herein by reference.

As used herein, “sequence complexity” (SqCx) is the nucleotide sequence of the combined total length in nucleotide (base) pairs of all of the different sequences in that genome. Because most prokaryotic genomes have relatively few repeats, the sequence complexity of a bacterial genome is essentially the same as its genome size (Britten and Kohne, 1968 Science, 161:529-540). Most eukaryotic genomes are populated by numerous repeat sequences, some of which may be repeated thousands or millions of times, and the sequence complexity is more difficult to determine. For a eukaryote, the sequence complexity is theoretically the combined length of all of the single-copy DNA sequences plus one copy of each repetitive sequence (e.g., a genome composed of 5000 copies of sequence A, 200 copies of sequence B, 30 copies of sequence C, and one copy each of sequences D-Z would have a sequence complexity of A+B+C+D+E . . . +Z bp) (Davidson and Britten, 1973 Quart. Rev. Biol., 48:565-613). However, the different copies of a repeat sequence typically exhibit nominal sequence divergence, making determination of sequence complexity difficult. Fortunately, from a Cot analysis one can determine the “kinetic complexity” of individual Cot components. As used herein, the term “kinetic complexity” is an estimate of sequence complexity determined from the reassociation rates, k, of individual Cot components (Britten et al., 1974 Meth. Enzymol., 29:363-405). The sum of the kinetic complexities of the components that constitute a genome is a good approximation of the sequence complexity of that genome. As used herein, the kinetic complexity of an isolated Cot component can be determined from a Cot analysis, or may be approximated through an estimate of the iteration frequency of individual elements in an isolated Cot component using techniques well known in the art.

As used herein, “dsDNA” refers to double-stranded DNA, and “ssDNA” refers to single-stranded DNA.

As used herein, “shotgun sequencing” refers to the sequencing of randomly selected genomic clones from a traditional, genomic library that contains DNA elements approximately in the frequencies at which they occur in the genomic DNA, exclusive of inadvertent biases associated with cloning or survival of recombinant bacteria that are well known to those of skill in the art.

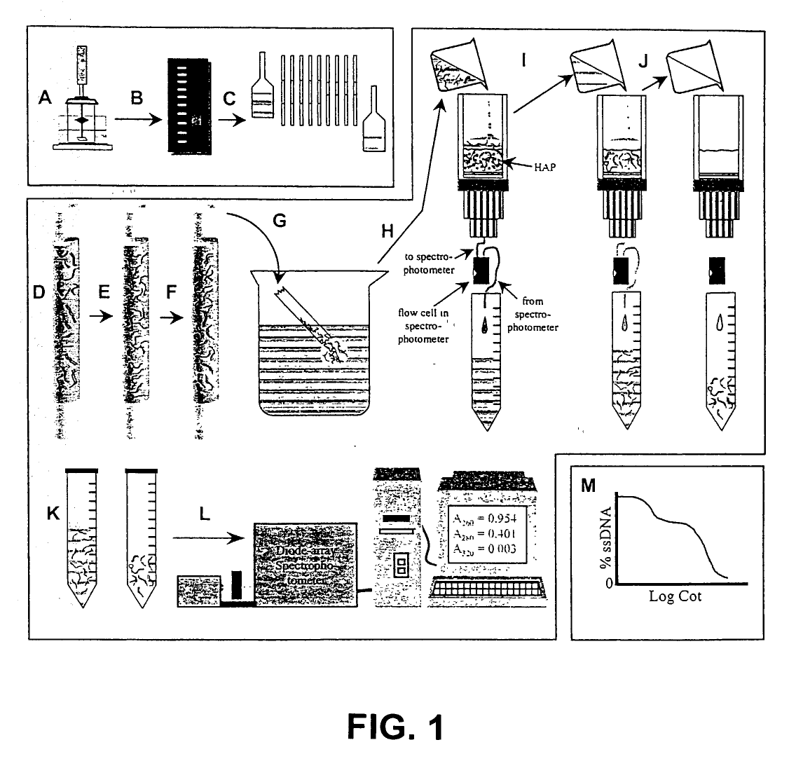

Hydroxyapatite (HAP) chromatography, especially column chromatography, is used to separate dsDNA and ssDNA molecules from mixtures containing both types of DNA molecule (Bernardi, 1971 Meth. Enzymology, 21: 95-139). Hydroxyapatite, Ca₁₀(PO₄)₆(OH)₂, is a compound that can discriminate nucleic acids with different secondary structures. Rigid, ordered structures (e.g., dsDNA) have a greater affinity for HAP than flexible, disordered structures (e.g., ssDNA). If the HAP column is equilibrated with low concentration (e.g., 0.03 M) sodium phosphate buffer (SPB), both dsDNA and ssDNA will stick to the column. At a SPB concentration of 0.12 M, ssDNA will preferentially elute from the column. When the SPB concentration is increased to 0.5 M SPB, any DNA remaining will be eluted from the column (FIG. 1; Britten et al., 1974 Meth. Enzymol., 29:363-405). While HAP chromatography is one preferred embodiment for fractioning genomic DNA, the invention contemplates the use of other methods for the fractionation of genomic DNA.

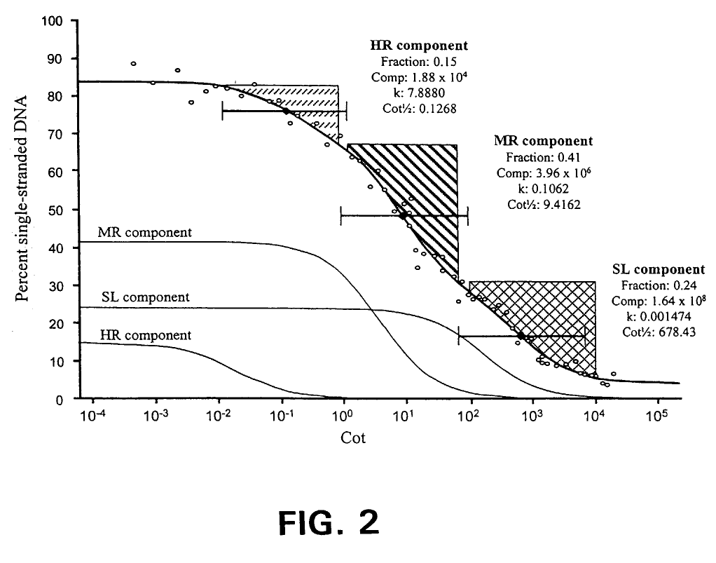

As used herein, “Cot analysis,” “Cot point,” and “Cot curve” refer to studies of the renaturation kinetics of nucleic acid molecules. When a solution of denatured genomic DNA is placed in an environment conducive to renaturation, the rate at which a particular sequence reassociates is proportional to the number of times it is found in the genome. This principle forms the basis of DNA renaturation kinetics (also called “Cot analysis”), a technique by which the redundant nature of eukaryotic genomes was first demonstrated (Britten and Kohne, 1968 Science, 161:529-540). In a typical renaturation kinetics study, samples of sheared genomic DNA are heat-denatured and allowed to reassociate to different “Cot values” (Cot value=the product of nucleotide concentration in moles per liter (C₀ or Co), reassociation time in seconds (t), and, if applicable, a factor based upon the cation concentration of the buffer (see Britten et al., 1974 Meth. Enzymol. 29:363-405)). For each sample, renatured DNA is separated from single-stranded DNA using hydroxyapatite (HAP) chromatography, and the percentage of the sample that has not reassociated (% ssDNA) is determined. The logarithm of a sample's Cot value is plotted against its corresponding % ssDNA to yield a “Cot point”, and a graph of Cot points ranging from little or no reassociation until reassociation approaches completion is called a “Cot curve” (see FIG. 2). Mathematical analysis of a Cot curve permits estimation of genome size, the proportion of the genome contained in the single-copy and repetitive DNA components, and the reassociation constant (k) and kinetic complexity (y) of each component. Interspecific comparisons of Cot data have provided considerable insight into the structure and evolution of eukaryotic genomes (e.g., Britten and Kohne, 1968 Science, 161:529-540; Davidson et al., 1975 Chromosoma, 51:253-259; Goldberg et al., 1975 Chromosoma, 51:225-251; Galau et al., 1976 In Molecular Evolution, F. Ayala, Ed. (Sunderland, MA: Sinauer Assoc.), pp. 200-224; Hake and Walbot, 1980 Chromosoma, 79:251-270; Geever et al., 1989 Theor. Appl. Genet., 77:553-559). As used herein, the term “renaturation kinetics based fractionation” refers to any method known now or in the future that allows for the isolation of the kinetic components of a sample of DNA. The phrase encompasses the use of Cot analysis, HAP chromatography, and any other method known now or in the future that allows separation of ssDNA and/or dsDNA from mixed populations of ssDNA and dsDNA. The term “renaturation kinetics based fractionation” does not require that Cot analysis be performed prior to the isolation of the kinetic components. In one embodiment, Cot analysis is performed prior to the isolation of the kinetic component or components. In another embodiment, Cot analysis is not performed prior to the isolation of the kinetic component or components, and the kinetic components are isolated based on estimates of their composition from other genomic data.

As used herein, a “Cot component” encompasses a group of genomic DNA sequences that exhibit similar reassociation properties. The sequence in a Cot component may appear as a mathematically distinct sigmoidal region of a complete Cot curve (Britten et al., 1974 Meth. Enzymol., 29:363-405). The similarity in reassociation characteristics between different sequences in a Cot component indicates that those sequences possess similar sequence complexities (i.e., they are found in similar copy numbers in the genome). Most higher eukaryotic genomes contain a fast reassociating (highly repetitive), a moderate reassociating (moderately repetitive), and a slow reassociating (usually single-copy) component.

As used herein, a “component fraction” refers to the proportion of a genome found in a particular Cot component.

As used herein, a “Cot½ value” is the point on the abscissa of a complete Cot curve at which half the DNA in a Cot component has reassociated (M sec).

As used herein, a “reassociation rate” (k) for a component is the inverse of its Cot½ (M⁻¹ sec⁻¹).

An “isolated Cot component,” as used herein, is a kinetic component fractionated from genomic DNA. Based on the results of a Cot analysis or other genomic data, HAP chromatography or any other method known now or in the future that allows isolation of ssDNA and/or dsDNA from mixed populations of ssDNA and dsDNA can be used to isolate the major kinetic components of a genome in a manner independent of sequence expression and/or methylation (e.g., Goldberg, 1978 Biochem. Genet., 16:45-68; Kiper and Herzfeld, 1978 Chromosoma, 65:335-351; Peterson et al., 1998 Genome, 41:346-356). Kinetic components fractionated from genomic DNA are referred to herein as isolated Cot components. S1 nuclease or mung bean nuclease, which preferentially degrade ssDNA, can be used to remove ssDNA from samples.

A “minicot analysis,” as used herein, is a Cot analysis performed using an isolated Cot component as the starting DNA source. Minicot analysis can be used to determine if the sequences in a component can be further divided into more than one subcomponent. In terms of Cot-based DNA isolation, minicot analysis and HAP chromatography can be used to fractionate genomes into more than one subcomponent encompassing more narrowly-defined ranges of sequence complexity than is possible in a Cot analysis using whole genomic DNA as the starting material.

An “isolated Cot subcomponent” is a fraction containing DNA from a specific complexity region of a Cot component. Subcomponents can be extracted from isolated Cot components using minicot analysis and/or any other applicable methods.

As used herein, “DNA resequencing techniques” (DRTs) are used to determine variants of a particular sequence after one variant (e.g., an allele) of a particular sequence has been elucidated. DRTs allow small differences such as base pair changes or small insertions/deletions between the sequenced (primary) variant and all other variants to be detected. DRTs include (but are not limited to) oligonucleotide microarray-based (DNA chip) hybridization analysis (Hacia, 1999 Nat. Genet., 21: 42-47), arrayed primer extension (Kurg et al., 2000 Genet. Test., 4: 1-7), denaturing high-performance liquid chromatography (Xiao and Oefner, 2001 Hum. Nut., 17: 439-474), and comparison of sequence traces derived from fluorescence-based automated DNA sequencing (Nickerson et al., 1997 Nucl. Acids Res., 25: 2745-2751).

As used herein, a “Cot library” is a genomic library prepared from an isolated Cot component or a Cot subcomponent, as defined above.

As used herein, a “Cot clone” is a clone from a Cot library.

As used herein, a “HRCot library” is a Cot library prepared from the highly repetitive (HR) component of a genome. In the present context, and in general, highly repetitive sequences are present in more than approximately 5000 copies per haploid genome.

As used herein, an “MRCot library” is a Cot library prepared from the moderately repetitive (MR) component of a genome. As used herein, and in general, moderately repetitive sequences are present at copy numbers from approximately 11 to approximately 4999 copies per haploid genome. In one embodiment, the moderately repetitive sequences are present at copy numbers more than approximately 10 copies per haploid genome.

As used herein, an “SLCot library” is a Cot library prepared from the single/low-copy (SL) component of a genome. Single/low copy number sequence components of a genome, in the context of the present invention, are present in from approximately 1 to approximately 10 copies per haploid genome. In a preferred embodiment, the single/low copy number sequence components are present in from approximately 1 to approximately 5 copies per haploid genome. In other preferred embodiments, the single/low copy number sequence components are present in fewer than approximately 9 copies, 8 copies, 7 copies, 6 copies, 5 copies, 4 copies, 3 copies, or 2 copies per haploid genome.

As used herein, an “FBCot library” is a Cot library prepared from the foldback fraction of a genome. As used herein, “foldback DNA” of a genome is composed of sequences that have formed duplexes at Cot values approaching zero. Such early renaturation is the result of intramolecular base pairing (i.e., pairing of complementary sequences on the same ssDNA molecule). Foldback DNA usually accounts for 1-15% of total reassociation. Because both repetitive and single/low-copy sequences may contain short regions in which foldback can occur, the sequence complexity of the fold-back cannot be predicted from a Cot analysis. With regard to this disclosure, isolated foldback DNA is not considered a kinetic component.

As used herein, a “genomic DNA library” is a library prepared from genomic DNA. The genomic DNA library need not contain a copy of every DNA sequence in a genome, however, in some instances it can contain at least one copy of every DNA sequence in a genome. As used herein, a “genome” is the collective DNA sequences found within the nucleus of a cell. As used herein, the term “genomic DNA” also refers to the DNA found within the nucleus of a cell.

As used herein, “IC DNA content” is the amount of DNA found within the nucleus of a gamete. It is usually expressed in base pairs or picograms. A typical somatic cell contains the 2C DNA content (two copies of each chromosome per cell).

The present invention contemplates sequencing a Cot clone from a Cot library or sequencing a Cot component to a depth determined based on the kinetic complexity of the isolated Cot component. The number or amount of clones required to be sequenced for the practice of the invention method will therefore vary as a function of the kinetic complexity of the Cot component, which will vary with the library and the genome. The kinetic complexity of an isolated Cot component can be determined from a Cot analysis, or may be estimated using techniques well known in the art. For example, the iteration frequency may be estimated based upon filter hybridization, in situ hybridization, Cot data from related species, or genome size. As used herein, the terms “depth,” “sequencing depth,” and “sequencing . . . to a depth” are interchangeable, and refer to the number of clones that must be sequenced to be confident of sampling a sufficiently representative number of nucleotides of a DNA sequence, a Cot component, a library, or a genome at least once. In mathematical terms, the number of clones (n) that must be sequenced in order to obtain a probability (p) of sequencing all the elements in a component at least once is predicted by the formula

n=ln(1−p)÷ln[1−(Z÷γ)]

where Z=mean insert size in bp and γ=the kinetic complexity (or an estimate of kinetic complexity) of the component in bp (Peterson et al., 2002, Genome Research, 12:795-807). For example, to achieve 80% probability of capturing all of the sequences in the MR component of a species with an MR kinetic complexity of 1×10⁶ bp and an MRCot clone mean insert size of 1000 bp, one would have to sequence

n=ln(1−0.8)+ln [1−(1000 bp÷11×10⁶ bp)]=1609 MRCot clones

Preferably, the statistical confidence level (i.e., probability expressed as a percentage) is greater than 10%, 20%, 30%, 40%, 50%, 60%, 70%, 80%, 85%, 90%, 91%, 92%, 93%, 94%, 95%, 96%, 97%, 98%, or 99%. In a preferred embodiment of the present invention, the depth of sequencing is proportional to the kinetic complexity of the isolated Cot component. In a preferred embodiment, the above methods are utilized to sequence more than approximately 0.5% of the genomic DNA of the organism, or of the isolated Cot component. In other preferred embodiments, the method is utilized to sequence more than approximately 1%, 2%, 3%, 4%, 5%, 6%, 7%, 8%, 9%, 10%, 15%, 20%, 25%, 30%, 35%, 40%, 45%, or 50% or more of the genomic DNA of the organism, or of the isolated Cot component.

As used herein, the term “recombinant nucleic acid molecule” refers to one which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions. Recombinant nucleic acid molecules can be made by ligating molecules of particular target Cot classes with vectors. In one embodiment, libraries of recombinant nucleic acid (DNA) molecules are made prior to sequencing efforts. In other embodiments of the present invention, the fractionated DNA is sequenced without first preparing a library from the DNA.

As used herein, “transformation” refers to any method known now or in the future that can be used to insert a foreign nucleic acid, such as a recombinant vector, into a cell, tissue, organ, or organism. The cell, tissue, organ, or organism into which the foreign nucleic acid has been introduced is considered “transformed” or “transgenic,” as is progeny thereof in which the foreign nucleic acid is present.

“Foreign” nucleic acids are nucleic acids that would not normally be present in the host cell, referring, in particular, to nucleic acids that have been modified by recombinant DNA techniques. The term “foreign” nucleic acids also includes host genes that are placed under the control of a new promoter or terminator sequence, for example, by conventional techniques.

As used herein, a “probe” is a nucleic acid molecule typically attached to a label or reporter molecule and is used to identify and isolate other sequences of interest. Probes comprising synthetic oligonucleotides or other polynucleotides may be derived from naturally occurring or recombinant single or double stranded nucleic acids or be chemically synthesized. Polynucleotide probes may be labeled by any of the methods known in the art, e.g., random hexamer labeling, nick translation, or the Klenow fill-in reaction. e.g., by the phosphoramidite method described by Beaucage and Caruthers, 1981 Tetra. Letts., 22: 1859-1862 or the triester method according to Matteuci et al., 1981 J. Am. Chem. Soc., 103: 3185, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

Large amounts of the recombinant DNA molecules may be produced by replication in a suitable host cell. Natural or synthetic DNA fragments coding for a protein of interest are incorporated into recombinant polynucleotide constructs, typically DNA constructs, capable of introduction into and replication in a prokaryotic or eukaryotic cell, especially Escherichia coli or Saccharomyces cerevisiae. Commonly used prokaryotic hosts include strains of Escherichia coli, although other prokaryotes, such as Bacillus subtilis or a pseudomonad, may also be used. Eukaryotic host cells include yeast, filamentous fungi, plant, insect, amphibian and avian species. Such factors as ease of manipulation, ability to appropriately glycosylate expressed proteins, degree and control of protein expression, ease of purification of expressed proteins away from cellular contaminants or other factors influence the choice of the host cell.

Appropriate promoter and/or vector sequences are selected so as to be functional in the recombinant host cell of choice. Examples of combinations of cell lines and expression vectors are described in Sambrook et al., 1989 Molecular Cloning, Second Edition, Cold Spring Harbor Laboratory, Plainview, N.Y.; Ausubel et al., (Eds.) 1997 Current Protocols in Molecular Biology, Greene Publishing and Wiley Interscience, New York; and Metzger et al., 1988 Nature, 334: 31-36. Many useful vectors for expression in bacteria, yeast, fungal, mammalian, insect, plant or other cells are well known in the art and may be obtained such vendors as Stratagene, New England Biolabs, Promega Biotech, and others. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, N.Y. (1983). While such expression vectors may replicate autonomously, they may less preferably replicate by being inserted into the genome of the host cell.

Expression and cloning vectors will likely contain a selectable marker. As used herein, the term “selectable marker” refers to a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that (a) confer resistance to antibiotics or other toxic substances, e.g., ampicillin, neomycin, methotrexate, etc.; (b) complement auxotrophic deficiencies; or (c) supply critical nutrients not available from complex media. The choice of the proper selectable marker will depend on the host cell; appropriate markers for different hosts are known in the art.

As used herein, “recombinant host cells” are those which have been genetically modified to contain an isolated or other recombinant DNA molecule, as described herein. The DNA can be introduced by any means known to the art which is appropriate for the particular type of cell, including without limitation, transformation, lipofection or electroporation.

The present invention can be used to isolate nucleotide sequences of interest. Additionally, it will be recognized by those skilled in the art that allelic variations may occur in particular DNA sequences. The skilled artisan will understand that a nucleotide sequence of interest can be used to identify and isolate additional nucleotide sequences that are related in sequence to the isolated DNA sequence of interest. The allelic variants may have a certain percent sequence identity with the nucleotide sequence of interest, and/or the allelic variants may hybridize to the nucleotide sequence of interest.

Hybridization procedures are useful for identifying polynucleotides with sufficient homology to a nucleotide sequence of interest. The particular hybridization technique is not essential to the subject invention. As improvements are made in hybridization techniques, they can be readily applied by one of ordinary skill in the art.

A probe and sample are combined in a hybridization buffer solution and held at an appropriate temperature until annealing occurs. Thereafter, the membrane is washed free of extraneous materials, leaving the sample and bound probe molecules typically detected and quantified by autoradiography and/or liquid scintillation counting. As is well known in the art, if the probe molecule and nucleic acid sample hybridize by forming a strong non-covalent bond between the two molecules, it can be reasonably assumed that the probe and sample are essentially identical, or completely complementary if the annealing and washing steps are carried out under conditions of high stringency. The probe's detectable label provides a means for determining whether hybridization has occurred.

Various degrees of stringency of hybridization can be employed for studies of cloned sequences isolated as described herein. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well know in the art, as described, for example in Keller, G. H., M. M. Manak, 1987 DNA Probes, Stockton Press, New York, N.Y., pp. 169-170, hereby incorporated by reference.

As used herein, moderate to high stringency conditions for hybridization are conditions, which achieve the same, or about the same, degree of specificity of hybridization as the conditions employed by the current inventors. An example of high stringency conditions are hybridizing at 68° C. in 5×SSC/5× Denhardt's solution/0.1% SDS, and washing in 0.2×SSC/0.1% SDS at room temperature. An example of conditions of moderate stringency are hybridizing at 55° C. in 5×SSC/5× Denhardt's solution/0.1% SDS and washing at 42° C. in 3×SSC. The parameters of temperature and salt concentration can be varied to achieve the desired level of sequence identity between probe and target nucleic acid. See, e.g., Sambrook et al. 1989 Molecular Cloning, Second Edition, Cold Spring Harbor Laboratory, Plainview, N.Y. or Ausubel et al., 1995 Current Protocols in Molecular Biology, John Wiley & Sons, NY, N.Y., for further guidance on hybridization conditions.

Specifically, hybridization of immobilized DNA in Southern blots with ³²P-labeled gene specific probes is performed by standard methods (Maniatis et al., 1982 Molecular Cloning, Cold Spring Harbor Laboratory, Plainview, N.Y.). In general, hybridization and subsequent washes are carried out under moderate to high stringency conditions that allow for detection of target sequences with homology to a particular nucleic acid molecule of interest. For double-stranded DNA gene probes, hybridization can be carried out overnight at 20-25° C. below the melting temperature (Tm) of the DNA hybrid in 6×SSPE 5× Denhardt's solution, 0.1% SDS, 0.1 mg/ml denatured DNA. The melting temperature is described by the following formula (Beltz et al., 1983 Methods of Enzymology, R.Wu, L, Grossman and K Moldave (Eds.) Academic Press, New York, pp. 266-285):

Tₘ=81.5° C.+16.6 Log[Na⁺]+0.41(+G+C) −0.61(% formamide) −600/length of duplex in base pairs.

Washes are typically carried out as follows: twice at room temperature for 15 minutes in 1×SSPE, 0.1% SDS (low stringency wash), and once at TM-20° C. for 15 minutes in 0.2×SSPE, 0.1% SDS (moderate stringency wash).

For oligonucleotide probes, hybridization is carried out overnight at 10-20° C. below the melting temperature (Tm) of the hybrid 6×SSPE, 5× Denhardt's solution, 0.1% SDS, 0.1 mg/ml denatured DNA. Tm for oligonucleotide probes is determined by the following formula: TM(° C.)=2(number T/A base pairs +4(number G/C base pairs) (Suggs et al., 1981 ICB-UCLA Symp. Dev. Biol. Using Purified Genes, D. D. Brown (Ed.), Academic Press, New York, 23:683-693).

Washes are typically carried out as follows: twice at room temperature for 15 minutes 1×SSPE, 0.1% SDS (low stringency wash), and once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used: Low, 1 or 2×SSPE, room temperature; Low, 1 or 2×SSPE, 42° C.; Moderate, 0.2×or 1×SSPE, 65° C.; and High, 0.1×SSPE, 65° C.

Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al., 1985 Science, 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes that can be used are known to those skilled in the art.

The percent sequence identity of two nucleic acids is determined using the algorithm of Karlin and Altschul, 1990 Proc. Natl. Acad. Sci. USA, 87:2264-2268, modified as in Karlin and Altschul, 1993 Proc. Natl. Acad. Sci. USA, 90:5873-5877. Such an algorithm is incorporated into the BLASTN and BLASTX programs of Altschul et al., 1990 J. Mol. Biol., 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al., 1997 Nucl. Acids. Res., 25:3389-3402. When utilizing BLAST and Gapped BLAST programs, the default parameters of the respective programs (BLASTN and BLASTX) are used. See http://www.ncbi.nih.gov for the default parameters.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol., 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning, Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### EXAMPLES

### Example 1

Plant Material

Herein, the production and characterization of genomic libraries derived from the three major Cot components of sorghum (Sorghum bicolor) DNA is described as a demonstration of the efficacy of CBCS. Sorghum was chosen for study because it has not been the subject of a Cot analysis, it has a 4000-6000 year history of cultivation (Kimber, 2000, In Sorghum: Origin, History, Technology, and Production, C. W. Smith and R. A. Frederiksen, Eds. (New York: John Wiley & Sons), pp. 3-98), it is currently one of the most agronomically-important species in the world, and its relatively small genome is a valuable “window” into the low-copy sequence diversity of closely related, large-genome crops such as maize and sugarcane (see Draye et al., 2001 Plant Physiol., 125:1325-1341).

Sorghum bicolor (L.) Moench (breeding line BTx623) DNA was used for Cot analysis, Cot cloning, and as a source of DNA in blotting experiments. For comparative purposes, Southern blots and colony blots containing DNA from Sorghum propinquum Kunth, a non-cultivated sorghum species crossed with BTx623 to make a detailed genetic map (Bowers et al., 2000 Plant Anim. Genome VIII Conf., www.intl-pag.org/pag/8/abstracts/pag8712.html); were probed with BTx623 DNA probes (see below).

### Example 2

Melting Curves and Cot Analysis

DNA isolation, preparation, and melting analyses were performed as described previously (Peterson et al., 1997 Plant Mol. Biol. Reptr., 15:148-153; Peterson et al., 1998 Genome, 41:346-356). Cot analysis was performed according to Peterson et al. (1998 Genome, 41:346-356) except that 0.5 M SPB was used to elute double-stranded DNA from HAP columns rather than 0.48 M SPB as described below. A least squares analysis of the Cot data was performed using the computer program of Pearson et al. (1977 Nucl. Acids Res., 4:1727-1734).

The steps involved in the Cot analysis are summarized in FIG. 1. This example is for a plant genome. Plant leaves were placed in an antioxidant medium and homogenized in a blender. The homogenate was filtered, plastids were preferentially lysed, and nuclei were pelleted by centrifugation. The pellet was largely free of contaminating organelles as determined by phase-contrast microscopy. DNA was isolated from purified nuclei using phenol/chloroform extractions coupled with proteinase and RNase digestions. The DNA was cut into pieces between 200-500 base pairs by high-speed blending. Fragment size was checked by agarose gel electrophoresis. Sheared DNA was precipitated, and aliquots of the DNA were dissolved in 0.03 M, 0.12 M, and 0.50 M sodium phosphate buffer (SPB) to produce solutions of known concentrations.

The DNA solutions were distributed into glass microcapillary tubes or glass ampoules so that each tube/ampoule contained 100 μg of DNA. The ends of the tubes/ampoules were sealed. Each of the tubes containing a known concentration of DNA were placed in boiling water to denature DNA duplexes. The tubes were then placed in a water bath set at a defined temperature. Renaturation was allowed to occur until the sample reached a specific Cot value (Cot value=the product of the sample's nucleotide concentration (moles of nucleotides per liter), its reassociation time in seconds, and an appropriate buffer factor based upon cation concentration).

Once the sample reached the desired Cot value, the end was broken off of the tube/ampoule, and the solution was blown into a 100-fold excess of 0.03 M SPB. The diluted sample quickly was loaded onto a hydroxyapatite (HAP) column equilibrated with 0.03 M SPB. At this buffer concentration, all DNA bound to the HAP. Once all of the solution entered the HAP, 0.12 M SPB was added causing single-stranded DNA (ssDNA) to elute. Eluant containing ssDNA was collected in a graduated polypropylene tube. After the ssDNA was collected, 0.50 M SPB was added to the column to elute double-stranded DNA (dsDNA). The volumes of the ssDNA eluant and the dsDNA eluant were determined. Exactly 0.9 ml of the centrifuged ssDNA eluant was mixed with 0.1 ml of aqueous 10 N KOH to denature any DNA duplexes. A sample of the dsDNA eluant was likewise denatured. The A₂₆₀ values (adjusted for light scatter at 320 nm) of the ssDNA/KOH mixture and the dsDNA/KOH mixture were determined.

For a particular Cot value, the percentage of ssDNA (% ssDNA) was calculated as follows:

[(Vss×Ass)×100]÷[(Vss×Ass)+(Vds×Ads)]=% ssDNA

where Vss=total volume of single-strand fraction, Vds=total volume of double-strand fraction, Ass=A₂₆₀ (adjusted for light scatter) for the KOH-denatured single-strand fraction, and Ads=A₂₆₀ (adjusted for light scatter) for the KOH-denatured double-strand fraction. The logarithms of Cot values ranging from essentially no renaturation to nearly complete renaturation were plotted against corresponding % ssDNA values to yield Cot points.

A graph of Cot points ranging from little or no reassociation until reassociation approaches completion is called a Cot curve. Because a DNA sequence reassociates at a rate that is directly proportional to the number of times it occurs in the genome, sequences that occur more than once in a genome (repetitive DNA) reassociate at lower Cot values than sequences found only once per genome (single-copy DNA). From an analysis of a Cot curve one can determine genome size, relative proportions of single-copy and repetitive sequences, the fraction of the genome occupied by each frequency component, and the complexity of the sequences in each frequency component.

Melting curves were generated for sheared sorghum DNA in 0.03, 0.12, and 0.5 M sodium phosphate buffer (SPB), and melting temperatures (Tm) for DNA in each buffer were determined using first-derivative analysis. The melting temperatures for sorghum DNA in 0.03, 0.12, and 0.5 M SPB were 75.1, 84.1, and 93.1° C., respectively.

For DNA dissolved in buffers with a monovalent cation concentration (Mmvc) between 0.01 and 0.2 M, the GC content of the DNA can be calculated using the formula %GC=2.44 (Tm-81.5-16.6 log Mmvc) (Mandel and Marmur, 1968 Methods Enzymol., 12:195-206). Consequently, the sorghum DNA samples in 0.03 M SPB (Na⁺=0.045 M) and 0.12 M SPB (Na⁺=0.18 M) result in %GC estimates of 38.9% and 36.5%, respectively. The average of these two values is 37.7%.

A Cot curve for Sorghum bicolor was prepared according to Peterson et al., 1998 Genome, 41:346-356 and analyzed using the computer program of Pearson et al., 1977 Nucleic Acids Res., 4:1727-1737. The analysis providing the lowest RMS (root mean square deviation) and Goodness of Fit values (0.02554 and 0.02712, respectively) was a three-component fit with no constrained variables. FIG. 2 shows the results of a sorghum Cot analysis. The top curve (open circles) illustrates a complete Cot curve, data analysis, and component isolation. A least-squares curve (thick line) was fit through the data points (open circles) using the computer program of Pearson et al., 1977 Nucl. Acids Res., 4:1727-1734. The curve consists of highly repetitive (HR), moderately repetitive (MR), and single/low-copy (SL) components characterized by fast, intermediate, and slow reassociation, respectively.

For each component, the following values were placed to the right of the component's general location in FIG. 2: Fraction=the proportion of the genome found in that component, Complexity=the length in nucleotide pairs of the longest non-repeating sequence, k =the observed reassociation rate in M⁻¹·s⁻¹, and Cot½=the value on the abscissa of the complete Cot curve at which half the DNA in the component has reassociated. Diamonds mark the positions on the complete Cot curve of the Cot½ values for HR, MR, and SL components. For a Cot component, 80% of the sequences in that component will renature in the “two Cot decade region” (TCDR) flanking the component's Cot½ value (brackets centered at Cot½ markers; Britten and Davidson, 1985 In Nucleic Acid Hybridisation, B. D. Hames and S. J. Higgins, Eds. (Washington D.C.: IRL Press), pp. 3-15.

Herein, the production and characterization of genomic libraries derived from the three major Cot components of sorghum (Sorghum bicolor) DNA is described as a demonstration of the efficacy of CBCS. In all Cot analyses, a certain fraction of the DNA forms duplexes even at Cot values approaching zero. Such early renaturation is thought to be due to base pairing between complementary sequences on the same DNA molecule (i.e., “fold-back” DNA; Britten et al., 1974 Meth. Enzymol., 29:363-405). As shown in FIG. 2, approximately 16% of the sorghum DNA had reassociated by the earliest Cot point (10⁻⁵ M·sec) and consequently was not included in the curve. No detectable reassociation was observed until a Cot value of approximately 0.02 M·sec.

Four percent of the sorghum DNA did not reassociate by the highest Cot value (20,000 M·sec). DNA that does not reassociate by such a high Cot value is thought to be damaged and incapable of binding to HAP (e.g., Kiper and Herzfeld, 1978 Chromosoma, 65:335-351; Stack and Comings, 1979 Chromosoma, 70:161-181; Leutwiler et al., 1984 Mol. Gen. Genet., 194:15-23.

The sorghum Cot curve consists of a fast, an intermediate, and a slow reassociating component. The complete Cot curve, renaturation profiles of the three Cot components, and the reassociation rate (k), Cot½ value, complexity, and genome fraction of each component are presented in FIG. 2. The Cot analysis is consistent with a sorghum genome size of approximately 700 Mb (in agreement with previous estimates) and the HR, MR, and SL components comprise 15, 41, and 24% of sorghum DNA, respectively.

In diploid organisms, the slowest reassociating component of a Cot curve represents single-copy DNA sequences. In such cases, genome size can be estimated by comparing the k value of the slow reassociating component to E. coli 's rate constant (k=0.22 M⁻¹·sec⁻¹) and DNA content (Zimmerman and Goldberg, 1977 Chromosoma, 59:227-252). The genome of E. coli is 4,639,221 base pairs (strain K12, substrain MG1655; Blattner et al., 1997 Science, 277:1453-1474). Assuming that the sorghum slow reassociating component (k 0.001474 M⁻¹·sec⁻¹) is composed of single-copy DNA, the estimated 1C genome size of sorghum would be

G=(4,639,221 bp×0.22 M⁻¹·sec⁻¹)÷0.001474 M⁻¹·sec⁻¹=6.92×10⁸ bp or 692 Mbp.

While this value is slightly smaller than the reported values based on Feulgen densitometry (753-837 Mbp, Laurie and Bennett, 1985 Heredity, 55:307-313) and flow cytometry (748-772 Mbp, Arumuganathan and Earle, 1991 Plant Mol. Biol. Reptr., 9:208-218), there is only a 7.5-17% difference between the Cot-based genome size and these previous estimates. Consequently, it is likely that the slow reassociating component is primarily single-copy DNA, and thus it is referred to as the single/low-copy (SL) component.

Assuming that the SL component has a repetition frequency of 1, the average repetition frequency of the DNA in the other components can be estimated by dividing their k values by the k value of the SL component (Hood et al., 1975 Molecular Biology of Eucaryotic Cells (Menlo Park, Calif.: W. A. Benjamin), pp. 56-61). The predicted repetition frequencies of sequences in the fast reassociating component and in the intermediate reassociating component are (7.8864÷0.001474=) 5350.3 and (0.1062 ÷0.001474=) 72.1, respectively. In light of their relative repetitiveness, the fast and intermediate reassociating components are hereafter referred to as the highly repetitive (HR) and moderately repetitive (MR) components.

### Example 3

CBCS is More Efficient than Shotgun Sequencing

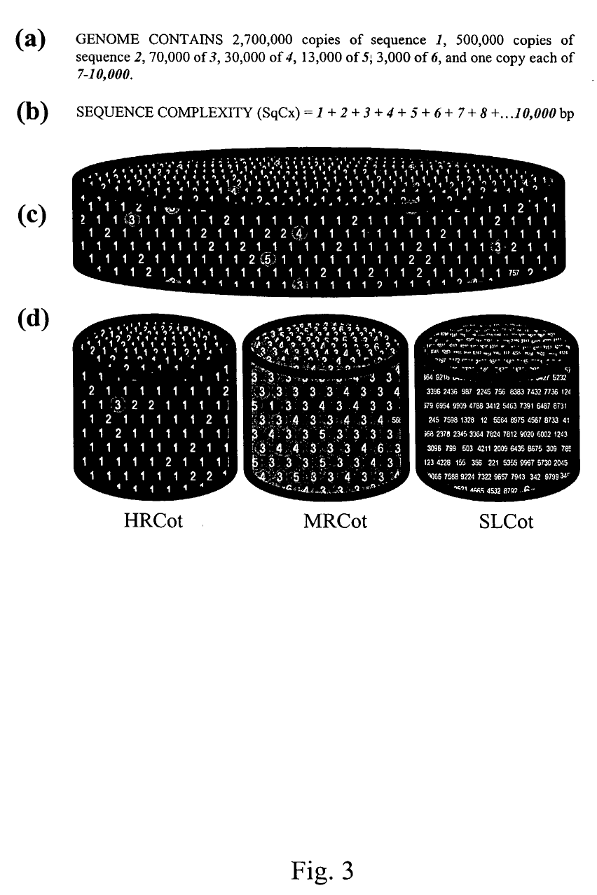

For many eukaryotes, CBCS is a far more efficient means of capturing the sequence complexity of an organism than “shotgun sequencing,” the sequencing of randomly selected genomic clones (see FIG. 3). In the shotgun approach, the genomic clones selected for sequencing have not been “pre-sorted” with regard to the relative sequence complexities of their inserts. Thus capture of the sequence complexity of a genome using shotgun sequencing is no more efficient than performing complete genome sequencing; the number of clones (N) that must be sequenced in order to attain a specific level of sequence complexity coverage (b) is N=β(G÷I) where G=genome size in bp and I=mean insert size of the clones. To obtain 10×sequence complexity coverage (β=10) using shotgun sequencing and a standard Sorghum bicolor genomic library (insert size=600 bp) would require sequencing of N=10 (760,000,000 bp÷600)=1.3×10⁷ clones.

In the present disclosure, sorghum (1C=760 Mbp) is used as a model genome, although the techniques described herein are applicable to any genome containing repetitive DNA. In CBCS, genomic DNA is fractionated into sequence complexity-based components that are subsequently used to construct Cot libraries. The number of Cot clones that must be sequenced in order to attain a specific level of sequence complexity coverage for a given component, a, can be estimated from that component's kinetic complexity (γₐ), i.e., Nₐ=β(γₐ÷I). In other words, the number of clones that must be sequenced to obtain a specific level of coverage for a Cot library is proportional to the kinetic complexity of the component from which the library was derived. The number of clones required to attain a specific level of sequence complexity coverage for an entire genome can be estimated from the sum of the kinetic complexities of the different components comprising that genome plus the number of base pairs in the fold-back region of the Cot curve (F), i.e., N=β[(γₐ+γ_{b}+γ_{c}, etc., +F)÷I].

It has been demonstrated that the sorghum genome contains a highly repetitive (HR), a moderately repetitive (MR), and a single/low-copy (SL) component with kinetic complexities of 1.9×10⁴ bp, 4.0×10⁶ bp, and 1.6×10⁸ bp, respectively. These three components, which were cloned to produce HRCot, MRCot, and SLCot libraries, comprise at least 80% of the sorghum genome. In the sorghum Cot analysis, 16% of reassociation was due to fold-back DNA. Thus the number of base pairs in the fold-back component is (0.16×760 Mbp=) 1.2×10⁸ bp. Assuming that the HRCot, MRCot, SLCot, and FBCot libraries have a mean insert size of 600 bp, the number of clones that would have to be sequenced to achieve 10×genome sequence complexity coverage (600 bp insert size) would be approximately N=10 ((1.6×10⁸ bp+4.0×10⁶ bp+1.9×10⁴ bp+1.2×10⁸ bp)÷600 bp)=4.7×10⁶. In other words, using CBCS one would only have to sequence approximately (4.7×106 . 1.3 x=0.28) 36% as many clones to achieve the same level of genome sequence complexity coverage as shotgun sequencing.

Similarly, the number of clones that would have to be sequenced in order to attain 1×coverage of that component's sequence complexity can be estimated by dividing the kinetic complexity of the component by the mean insert size of clones in the component. For the HRCot library having a mean insert size of 200 bp, (1.88×10⁴ bp+200 bp=) 94 clones would need to be sequenced to attain 1×sequence complexity coverage. However, if the HR component fragments had been concatenated into groups of three prior to cloning, the number of HR clones that would need to be sequenced to attain IX coverage would be (1.88×10⁴ bp . 600 bp=) 31. Likewise, 1×component coverage could be achieved for sorghum MRCot and SLCot libraries (with 600 bp inserts) by sequencing 6600 clones and 273,333 clones, respectively. The sorghum HR, MR, and SL components have a combined sequence complexity of 1.68×10⁸ bp (of which 98% is localized in the SL component) and collectively account for 80% of the DNA in the sorghum Cot analysis. The remaining 20% of the genome is divided between foldback DNA (16%) and damaged (unannealable) sequences (4%).

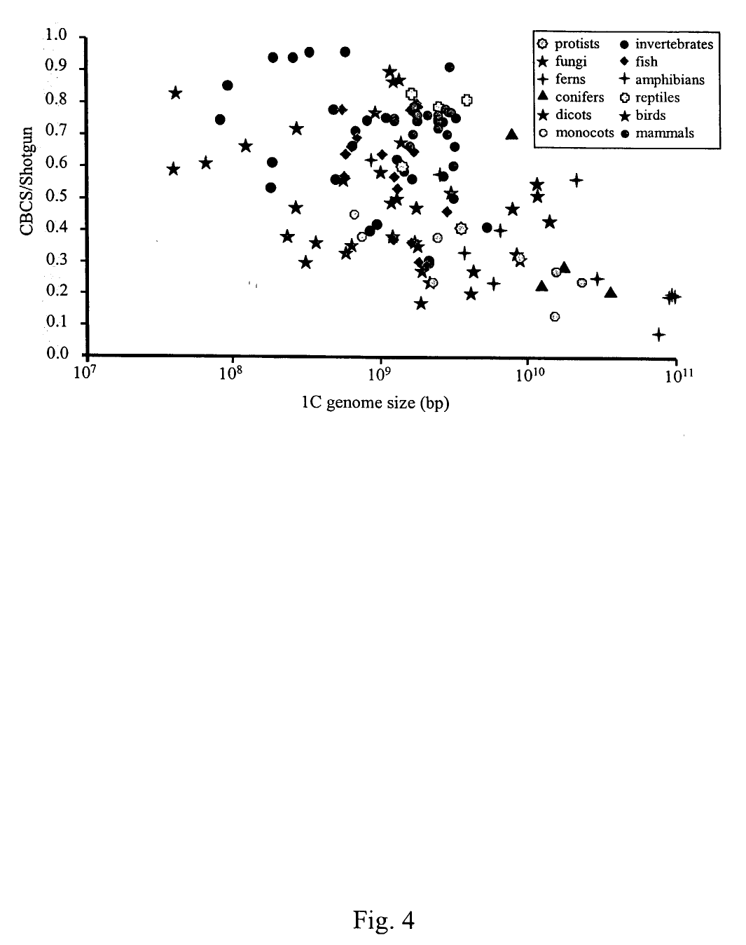

Note that the foldback DNA has been treated as if its complexity were equal to the number of base pairs in the foldback fraction. This is most certainly not the case—the majority of foldback sequences are probably repetitive in nature (Davidson et al., 1971 Dev. Biol., 25: 445-463). Presumably strategies to minimize sequencing of foldback clones could be developed, thus further increasing the relative advantage of CBCS over shotgun sequencing. Although most foldback DNA is probably repetitive in nature, some foldback sequences may be single/low-copy DNA; likewise the foldback fraction may contain some sequences not represented in the HR, MR, and/or SL components. Consequently, foldback DNA is presumably a source of useful sequence information, and genomic libraries prepared from isolated foldback DNA (i.e., FBCot libraries) should be considered when trying to capture the sequence complexity of an entire genome. To be fairly secure of retrieving the useful sequence information from the foldback fraction, it can be assigned a “kinetic complexity” equal to the number of base pairs it contains. In sorghum, the foldback fraction contains (0.16×760 Mbp=) 1.2×10⁸ bp of DNA. Adding the combined HR, MR, and SL kinetic complexity (1.68×10⁸ bp) to the number of base pairs in the foldback fraction (1.2×10⁸ bp) and dividing by the average insert size (e.g., 600 bp) yields the total number of clones that would need to be sequenced to attain 1×sequence complexity coverage (or a close approximation thereof) of the entire sorghum genome, i.e., ((1.68×10⁸ bp+1.2×10⁸ bp)+600 bp=) 480,000. Undoubtedly sequencing of 480,000 clones to obtain 1×complexity coverage of the sorghum genome would be a significant undertaking. However, obtaining comparable coverage using the “shotgun approach” (i.e., sequencing of randomly-selected genomic clones) would require sequencing of roughly (760 Mb ÷600 bp=) 1,300,000 clones. In other words, DNA sequencing requirements could be reduced by roughly three fold if CBCS rather than shotgun sequencing was employed to “capture” sorghum's sequence complexity. The relative advantage of CBCS over shotgun sequencing would be even more pronounced for species possessing genomes with higher proportions of repetitive DNA, but in all cases it can be quantitated in advance of initiating sequencing (FIG. 4).

CBCS provides the greatest advantage with genomes composed primarily of repetitive DNA. The sequence complexity of many plant and several amphibian genomes can be captured using less than one-third the number of clones required using the shotgun approach (FIG. 4). FIG. 4 compares the efficiency for CBCS versus shotgun sequencing. For each species, the number of Cot clones that would need to be sequenced to attain a specific level of sequence complexity coverage has been divided by the number of randomly selected clones (i.e., “shotgun clones”) that would have to be sequenced in order to attain the same level of coverage. The “Cot/shotgun” ratio for each species was determined using the following formula: Ratio=(γ_{CR}+γ_{SL}+F)+G where γ_{CR}=the sum of the kinetic complexities of all repetitive components in the genome, γ_{SL}=the kinetic complexity for the single-copy or single/low-copy component, F the base pair content of the foldback fraction, and G=1 C genome size in bp. Resulting values have been plotted against genome size. Cot and genome size information used in generating this figure can be found at www.plantgenome.uga.edu/CBCS and in the scientific literature.

For Allium cepa, the table onion, 26 million clones with a mean insert length of 600 bp must be sequenced to attain 1×sequence complexity coverage using the shotgun approach to cloning and sequencing. CBCS reduces this number to roughly 3.45 million, an 87% reduction. These calculations account for the approximately 7.2% foldback DNA in the A. cepa genome. CBCS thus opens the door to capturing the sequence complexity of a wide range of flora and fauna for which shotgun sequencing is prohibitively costly, frightfully tedious and very labor-intensive.

### Example 4

Cloning of Cot Components

Briefly, all the double-stranded DNA within a component's TCDR was isolated except for the areas that overlap the TCDRs of other components. In FIG. 2, the regions marked by upward vertical dashes, diagonal stripes, and crosshatching delimit areas of the curve used in HRCot, MRCot, and SLCot library construction, respectively. Note that the area isolated for use in constructing the SLCot library extends a short way past the right end of the TCDR for the SL component; this is presumably not a problem as any double-stranded DNA in the region to the right of the SL component TCDR is likely to be single-copy. The curves at the lower portion of the graph show the predicted individual renaturation profiles of the HR component, MR component, and SL component.

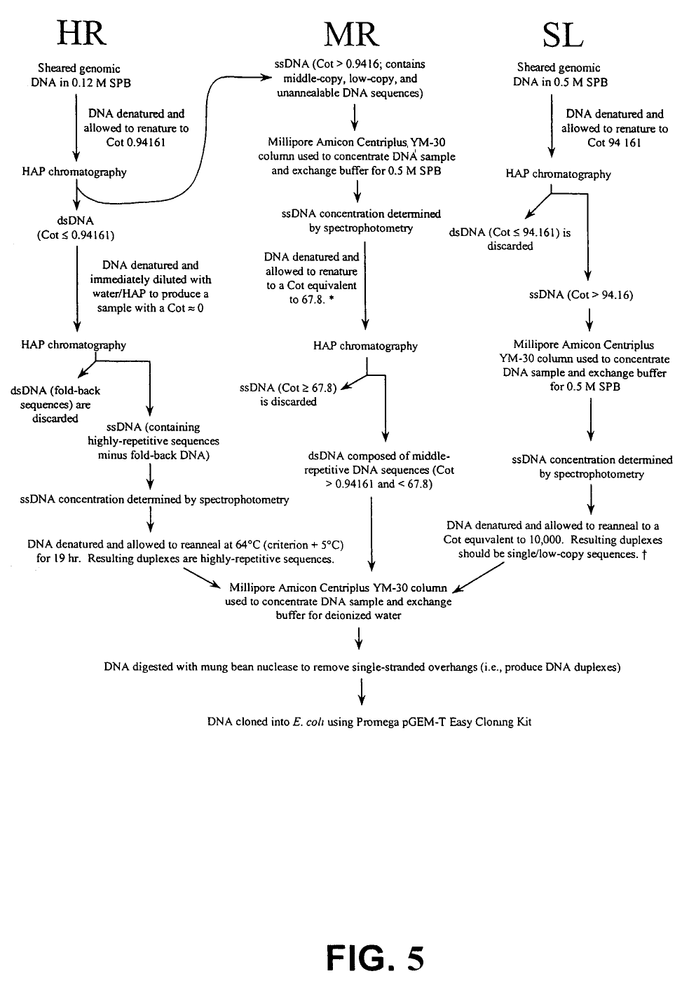

Highly repetitive (HR), moderately repetitive (MR), and single/low-copy (SL) DNA components of the Cot curve were prepared for cloning as outlined in FIG. 5. The sections of the Cot curve used for cloning (i.e., roughly the two Cot decade regions flanking the Cot½ value of each component) are shown in FIG. 5. DNA sample concentrations were determined using KOH-denaturation and spectrophotometry as described by Peterson et al., 1998 Genome 41:346-356.

Isolated Cot components were digested with mung bean nuclease (Promega, Madison, Wis.) to remove single-stranded DNA overhangs in accordance with the manufacturer's instructions, and the resulting blunt-ended molecules were cloned into E. coli (JM109) using the Promega pGEM®-T Easy cloning kit (Cat. No. A1380). The HRCot, MRCot, and SLCot libraries were plated onto selective media, and positive clones were transferred via sterile toothpicks into freezing medium in 96-well microtiter plates. In total, four plates of HRCot, five plates of MRCot, and six plates of SLCot clones were obtained. Cot libraries were replicated using a hand-held 96-pin replicator and stored at −80° C. (see Peterson et al., 2000 J. Agric. Genomics 5, www.ncgr.org. for details). Each clone was named based upon the library, plate, row, and column in which it was found (e.g., HRCot3A10=HRCot library, plate 3, row A, column 10).

### Example 5

DNA Sequencing

Plasmids were isolated from Cot clones using an alkaline lysis method with modifications made for the 96-well plate format (Marra et al., 1997 Genome Res., 7:1072-1084). Cycle sequencing reactions were performed using the BigDye Terminator® Cycle Sequencing Kit Version 2 (Applied Biosystems, Foster City, Calif.) and an MJ Research (Watertown, Pa.) PTC-100 thermocycler. Finished cycle sequencing reactions were filtered through Sephadex filter plates (Krakowski et al., 1995 Nucleic Acids Res., 23:4930-4931) directly into Perkin-Elmer MicroAmp Optical 96-well reaction plates. Sequencing was performed using an ABI 3700 automated DNA Analyzer. ABI sequencer trace data was evaluated using the programs PHRED, CROSS_MATCH, and PHRAP (see www.phrap.org for additional information). Only clones with a Ph/Pr value >16 over 300 continuous base pairs and insert sequences ≧50 bp in length were used in sequence analyses. Sequences and descriptive information regarding each of these “quality clones” is available in the GenBank dbGSS Database (HRCot clones, AZ921847-AZ922099; MRCot clones, AZ922100-AZ922508; SLCot clones, AZ922509-AZ923007).

### Example 6

Sequence Analysis

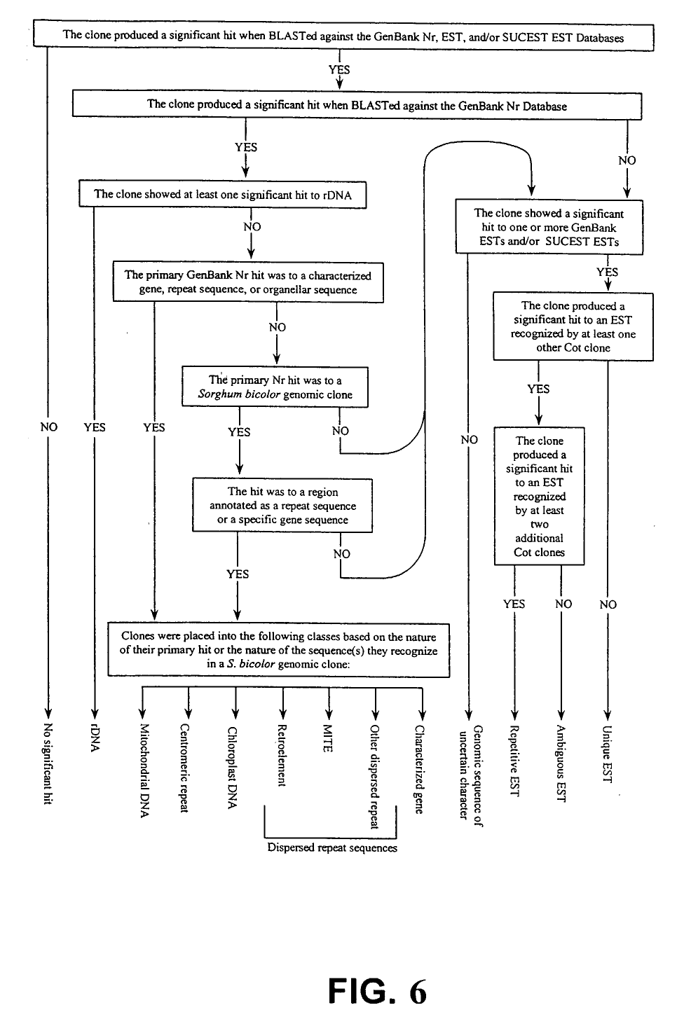

The sequence of each Cot clone was compared to sequences in the GenBank Nr and EST Databases (http://www.ncbi.nlm.nih.gov), and the SUCEST Sugarcane EST Database (http://sucest.lbi.dcc.unicamp.br/en/) using standard BLAST (blastn) protocols (Altschul et al., 1997 Nucleic Acids Res., 25:3402). Based on the nature of the hits obtained, each Cot clone insert sequence was placed into a single descriptive “BLAST category” according to the scheme shown in FIG. 6.

HRCot, MRCot, and SLCot libraries were generated from isolated Cot components. The relative iteration of the insert DNA in the three Cot libraries was examined by comparing the intensity with which Cot probes hybridized to replica Southern blots of sorghum genomic DNA. The average intensity of hybridization to the blots incubated with HRCot probes was 43,067 cpm (±6248) while the average values for the MRCot and SLCot blots were 3783 cpm (±1419) and 1377 cpm (±253), respectively.

A total of 384 HRCot, 480 MRCot, and 576 SLCot clones were sequenced, of which 253, 409, and 499 (respectively) met the sequence quality criteria (i.e., Ph/Pr value >16 over 300 continuous bases, high quality insert sequence ≧50 bp). The sequence complexity of a Cot component is the combined total length in nucleotide pairs of the different DNA sequences in that component (Britten et al., 1974 Meth. Enzymol., 29:363-405). In theory, each repeat sequence is represented only once in the calculation of the component's complexity (e.g., a component composed of 5000 copies of sequence A, 200 copies of sequence B, and 30 copies of sequence C would have a complexity of A+B+C bp). Because most prokaryotic genomes are relatively devoid of repetition, the complexity of a bacterial genome is essentially the same as its genome size (Britten and Kohne, 1968 Science, 161:529-540). In eukaryotes, the complexity of the single-copy component of a genome is equal to the number of base pairs in the single/low copy sequence component while repetitive DNA components have complexities inversely proportional to their repetitiveness.

For a genomic library containing random pieces of genomic DNA (e.g., a typical BAC library), the probability (p) that the library contains a particular sequence of interest can be estimated using the formula

p=1−e^{n·ln[1-(Z÷G)]}

where n=the number of clones in the library, Z=mean insert size in bp, and G=1C genome size in bp (Paterson, 1996 In Genome Mapping In Plants, A. H. Paterson, Ed. (San Diego: Academic Press), pp. 55-62). However, for a library containing DNA from a particular Cot component, the probability of finding a given sequence from that component can be estimated by replacing genome size in the equation above with the component's sequence complexity. The 253 HRCot, 409 MRCot, and 499 SLCot quality clones have an average insert size of approximately 200 base pairs, and the complexities of the HR, MR, and SL components are 1.88×10⁴, 3.96×10⁶, and 1.64×10⁸ base pairs, respectively (FIG. 4). Assuming that the sorghum Cot libraries are representative of the components from which they were derived, the likelihood of finding a particular HR component sequence among the HRCot quality clones would be estimated as follows:

p=1−e^{253·ln[1-(200÷18,800)]}=0.93.

In other words, the probability that all the HR sequences in the sorghum genome are found within the 253 HRCot quality clones is roughly 93%. The probability that the MRCot library contains all MR component sequences is 0.02 (i.e., 2.0%) while the probability that the SLCot library contains all the SL component sequences is 6.1×10⁻⁴ (0.06%). Obviously, many more clones are required to construct representative libraries from high-complexity Cot components than from low-complexity components.

The (253+409+499=) 1,161 “quality clones” were BLASTed against the GenBank Nr (non-redundant), GenBank EST, and SUCEST Sugarcane EST (http://sucest.lbi.dcc.unicamp.br/en/) Databases. For each quality clone, only bit scores (S′) of 55.44 or greater were deemed significant and used in characterizing the clone. Unlike “E values” (E) commonly used to compare the quality of hits, bit scores provide a means of comparing the significance of database hits independent of database and query size (see www.ncbi.nlm.nih.gov for details). For a database of 3.5 billion nucleotides (slightly larger than the effective size of the GenBank Nr and EST Databases at the time of sequence analysis) and an effective query length of 159 nucleotides, a bit score of 55.44 is roughly equivalent to an E value of 1×10⁻⁵. For a given quality clone, the term “primary hit” was used to indicate the database sequence (if any) showing the highest significant homology to that clone.

There are limitations inherent in EST data. Approximately 41.9% of the HRCot, 50.0% of the MRCot, and 11.8% of the SLCot clones with significant hits to EST sequences also show primary GenBank Nr hits to known repeat sequences (Table 1). While certain repetitive DNAs in the EST databases are contaminants (e.g., rDNA, chloroplast DNA), others represent transcribed portions of repetitive elements (e.g., retrotransposon genes). As there are many repetitive DNA sequences not in the GenBank Nr Database, it is believed, without wishing to be bound by any particular theory, that some of the “EST-hit clones” (ECs) without significant homology to Nr database repeats (i.e., ^{Nr}-ECs) also represent repetitive DNA. Recognition of the same database entry by multiple clones is one means by which clone repetitiveness has been estimated (e.g., Bureau et al., 1996 Proc. Natl. Acad. Sci. USA, 93:8524-8529; Rabinowicz et al., 1999 Nature Genet., 23:305-308). Consequently, the significant EST hits for each of the 308 ^{Nr-}ECs were compared to the significant EST hits of all other ECs (including other ^{Nr-}ECs). If sorghum has roughly 25,000 non-repetitive gene sequences, like Arabidopsis (Arabidopsis Genome Initiative, 2000), and the 308 ^{Nr-}ECs are single-copy sequences, the average expected number of hits by an ^{Nr-}EC to any one of the hypothetical sorghum genes is (308÷25,000=) 0.0123. The probability of multiple ^{Nr-}ECs recognizing a particular “single-copy EST” (>>gene) sequence by chance can be roughly estimated using the Poisson probability distribution function

$$P ( X ) = \frac{{μ}^{X}}{{e}^{μ} X !}$$

where P=probability, X=number of occurrences, and μ=is the population mean number of occurrences in a unit of space or time (Zar, 1996 Biostatistical Analysis (Upper Saddle River, NJ: Prentice Hall)). If μ=0.0123 (see above), the probabilities of two, three, four, and five ^{Nr-}ECs recognizing the same single-copy EST by chance are 7.5×10⁻⁵, 3.1×10⁻⁷, 9.5×10⁻¹⁰, and 2.3×10⁻¹², respectively. If sorghum has more than 25,000 genes, the likelihood of multiple clones having single-copy EST hits in common would be even lower. However, as shown in Table 1, 140 of the 308 ^{Nr-}ECs (i.e., 45.5%) show significant homology to at least one EST(s) identified by other Cot clones. This finding clearly indicates that many of the ^{Nr-}ECs clones contain repetitive sequences. Interestingly, 88.1% of the ^{Nr-}ECs in the SLCot library share no significant EST hits with other ECs while only 30.4% of the HRCot and 24.2% of the MRCot ^{Nr-}ECs possess EST hits not shared by other ECs.

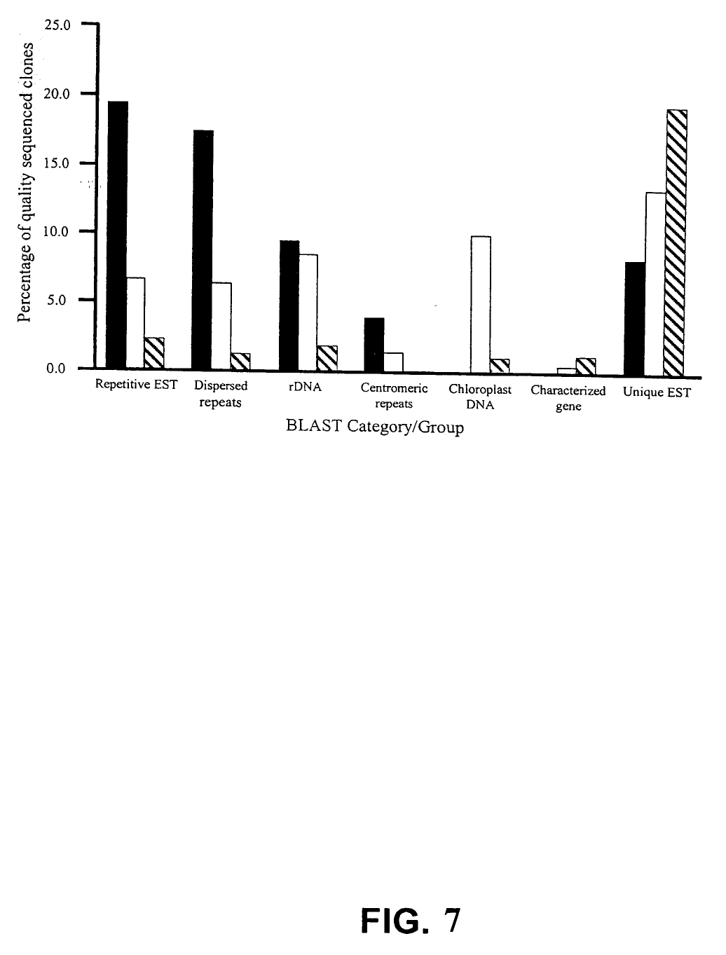

Each Cot clone was placed into a single descriptive category (“BLAST category”) based upon the scheme shown in FIG. 6. Because of the complexity associated with evaluating EST hits (see above), GenBank Nr Database hits were given priority in the classification scheme with EST hits used only to categorize clones without significant Nr hits or with Nr hits to genomic sequences of unknown character. Results of category assignment and a list of characterized gene and repeat sequences recognized by various Cot clones are given in Table 2. An overview of the data is shown in FIG. 7.

All three libraries possessed more clones in the “no significant hit” BLAST category than any other. Roughly 70% of the SLCot clones showed no significant database hits while about 50% of the MRCot clones and 35% of the HRCot clones fell into the “no significant hit” category.

HRCot hits were primarily to repetitive DNA sequences (Lapitan, 1992 Genome, 35:171-181; Bennetzen et al., 1998 Proc. Natl. Acad. Sci. USA, 95:1975-1978; Heslop-Harrison, 2000 Plant Cell, 12:617-635) including retrotransposons and other dispersed repeat sequences, rDNA sequences, and sorghum centromeric repeat sequences. The relative percentage of clones showing homology to repetitive ESTs was considerably higher for the HRCot library (19.4%) than the other two libraries (6.6% for MRCot, 2.2% for SLCot). None of the HRCot clones produced a significant hit to a characterized gene sequence, and the percentage of single/low copy EST clones in the HRCot library was much lower than corresponding values for the MRCot and SLCot libraries (Table 2 and FIG. 7).

The SLCot library showed the highest percentage of hits to characterized gene sequences and single/low copy EST Cot clones of the three Cot libraries. No centromeric sequences were detected, and only 6.2% of the SLCot clones fell into one of the other repeat sequence categories.

The MRCot library showed intermediate levels of repeat sequences and single/low copy sequences. With regard to low-copy sequences, the percentage of MRCot sequences in the single/low copy EST category was roughly the mean of the corresponding values for the HRCot and SLCot libraries (FIG. 7). Of the characterized gene sequences detected in the Cot libraries, 25% were found in the MRCot library, and the remaining 75% were found in the SLCot library. Although the HRCot library had the greatest fraction of clones with homology to known repeats (Table 2), some repeat sequences that are believed to be of moderate iteration were more abundant in the MRCot library. For example, clones with homology to the retroelement Leviathan were three times as common in the MRCot library than the HRCot library. Likewise, sequences with homology to retrotransposon genes/pseudogenes were generally limited to MRCot clones (Table 2). Of particular note, 10% of the MRCot sequences correspond to chloroplast DNA, which was presumably a contaminant in the nuclear DNA isolation process (FIG. 7). However, chloroplast sequences were detected in less than one percent of the SLCot clones and were not detected in any of the HRCot clones. While chloroplast DNA was not a desired end product of Cot cloning, the observation that chloroplast sequences are almost exclusively limited to MRCot clones neatly illustrates the “two Cot decade” principle used in the isolation of individual Cot components, i.e., 80% of the copies of a given DNA sequence are contained within a span of two Cot decades (Britten and Davidson, 1985 In Nucleic Acid Hybridisation, B. D. Hames and S. J. Higgins, Eds. (Washington D.C.: IRL Press), pp. 3-15; FIG. 3). Based on the Cot curve, the MR component constitutes 41% of the genome, but if a tenth of this component is actually chloroplast DNA, the percentage of the genome found in the MR component may be closer to 37%.

Of the 1161 Cot clone sequences used in sequence analysis, only one clone showed a significant primary hit to a mitochondrial DNA sequence. This clone (SLCot4GO5) appears to contain a portion of the Sorghum bicolor F0-F1 ATPase alpha subunit gene (GenBank AJ278690).

One of the largest continuous sorghum DNA sequences in the GenBank Nr Database is a 126 kb BAC clone containing the 22 kDa kafirin cluster (GenBank AF061282; Llaca, Lou, and Messing, unpublished results). A total of 15.2% of the HRCot clones, 2.4% of the MRCot clones, and 1.0% of the SLCot clones showed primary hits to this BAC. Interestingly, (34/39=) 87.1% of the HRCot and (7/7=) 100% of the MRCot primary hits to the kafirin cluster BAC were localized within a 7377 bp sequence found only once in the BAC (bases 127,895-135,271). None of the SLCot hits to the kafirin cluster BAC recognized the 7377 bp sequence. Although the 7377 bp sequence represented only 4.5% of the bases in the kafirin cluster BAC, it accounted for 13.4% of all primary HRCot hits, making it the most frequently cloned Cot sequence in the S. bicolor genome.

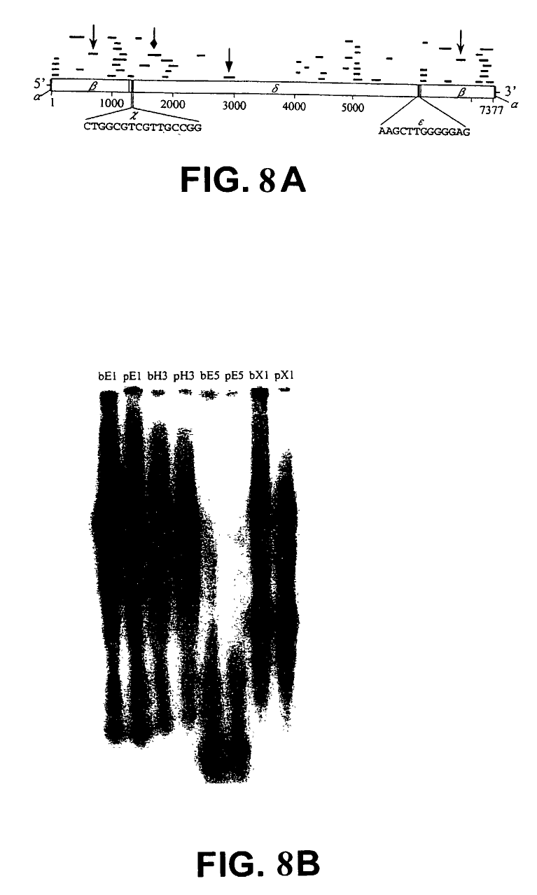

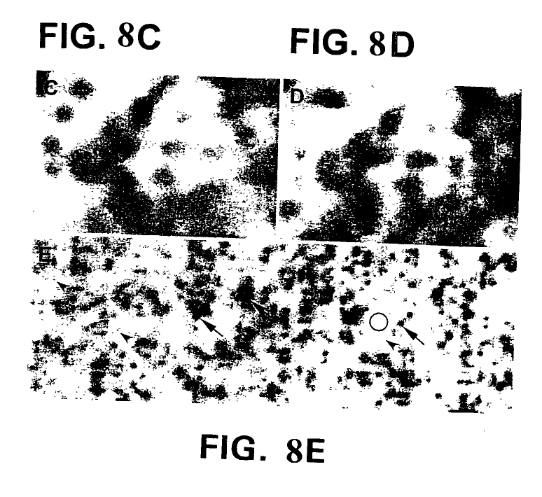

In their annotation of the kafirin cluster BAC, Llaca, Lou, and Messing deemed the 7377 bp sequence a “retroelement”. Although they named five other sorghum retroelements (Retrosor-1, Retrosor-2, Retrosor-3, Retrosor-4, and Retrosor-5), Messing and colleagues did not name the 7377 bp retroelement sequence. The present study of the sequence likewise suggests that this sequence is a retroelement (see FIG. 8A), and with the support of Messing, the sequence was named Retrosor-6. Retrosor-6 possesses no large open reading frames (ORFs) although nucleotide-protein BLAST (blastx) results indicate that it shares limited homology to both an ORFI polyprotein (S′=43.9 bits) of the gypsy-type retroelement Athila (Pelissier et al., 1995 Plant Mol. Biol., 29:441-452) and to a putative Arabidopsis pol protein (S′=42.4 bits). The apparent absence of gag and env genes and the limited homology to known pol sequences suggest that the copy of the retroelement found in the kafirin cluster is no longer capable of autonomous replication.

To examine the abundance and dispersal pattern of Retrosor-6 in the genome of S. bicolor and to check for its presence in the wild species S. propinquum, a Cot clone containing 190 bp of the Retrosor-6 sequence was radiolabeled and used to probe a Southern blot containing restriction-digested S. bicolor and S. propinquum DNA. FIG. 8B shows that the Retrosor-6 hybridization pattern for both sorghum species was essentially the same, consisting of a few dark bands within a smear of hybridization signal.

While most of the retroelement showed numerous Cot clone hits, the region between bases 2000 and 4000 had only two hits. To explore whether this region diverged more rapidly than other parts of Retrosor-6, high-density BAC grids from both sorghum species were probed with a Cot clone containing part of the Retrosor-6 LTR sequence (e.g., FIG. 8C) while duplicate copies of the grids were probed with a sequence from the 2-4 kb region of the retroelement (FIG. 8D). When the autoradiograms for the LTR region and the 2-4 kb region were digitally aligned and compared, only minimal differences could be detected in the hybridization patterns for a particular species (compare FIGS. 8C and 8D).

To estimate the copy number of Retrosor-6 in the genomes of S. bicolor and S. propinquum, the BAC grids probed with the Retrosor-6 LTR sequence were analyzed using a densitometer (see FIG. 8E). The grid densitometry results indicates that there are approximately 6275 copies of Retrosor-6 in the S. bicolor genome and 6748 copies in the S. propinquum genome (see Table 3). Assuming an average size for the retroelement of 7377 base pairs, Retrosor-6 accounts for approximately 6.0% and 6.3% of genomic DNA in S. bicolor and S. propinquum, respectively (Table 3).

Of note, two of the randomly-selected HRCot clones hybridized to Southern blots were later shown to contain portions of Retrosor-6. One clone that contained part of the Retrosor-6 LTR, HRCot2G1 1, produced the highest level of hybridization of any of the randomly-selected Cot clones, with a specific activity of 10,000 cpm. The second clone carrying part of the internal sequence of Retrosor-6, HRCot3BO1, resulted in a hybridization intensity of 5000 cpm, i.e., half that of the clone containing the LTR sequence.

Cot clones were BLASTed against a local database containing sequences of approximately 2000 molecular markers from a high-density sorghum molecular map based on RFLP segregation in the progeny of a cross between S. bicolor and S. propinquum (Chittenden et al., 1994 Theor. Appl. Genet., 87:925-933; Draye et al., 2001 Plant Physiol., 125:1325-1341). Fourteen Cot clones contained inserts with significant homology (S′≧76.28) to markers on the molecular map (see Table 4 for details).

The Retrosor-6 sequence (bases 127,895-135,271 of GenBank AF061282) was compared to data in the GenBank Nr Database using standard blastn (nucleotide query—nucleotide database) and blastx (nucleotide query—protein database) programs (Altschul et al., 1997 Nucleic Acids Res., 25:3402).

There is bias in the sorghum Cot libraries with regard to representation of methylated DNA. E. coli possesses three endonuclease systems that preferentially restrict methylated DNA; McrA, McrBC, and Mrr. These restriction systems do not cleave DNA that has been methylated by the bacterium's endogenous methylase systems (Redaschi and Bickle, 1996 In Escherichia coli and Salmonella: Cellular and Molecular Biology, 2nd Edition, F.C. Neidhardt, Ed. (Washington, D.C.: ASM Press), pp.773-781). In preparing the sorghum Cot libraries, the Promega pGEM-T Easy cloning kit and the accompanying host strain Escherichia coli JM 109 were used. While E. coli JM 109 lacks functional McrA and Mrr restriction systems (it is mcrA⁻, mrr⁻), it does possess a functional McrBC protein (mcrBC⁺). The McrBC protein cleaves DNA sequences with the following configuration: 5′-Pu^{m}CN₄₀₋₈₀Pu^{m}C-3′ (Pieper et al., 1997 J. Mol. Biol., 272:190-199). Consequently, it is possible that certain methylated (presumably highly repetitive) sequences from sorghum are under-represented in one or more of the Cot libraries due to preferential restriction by the McrBC system. However, it is believed that the relatively small size of the Cot clone inserts (˜100-400 bp) and the relatively large size of McrBC recognition sites (≧44 bp) substantially decreased possible effects of McrBC during cloning. The limited effect of the cloning host McrBC genotype on sorghum Cot library construction is consistent with the observation that the highest proportion of HRCot clones showing significant hits to the GenBank Nr Database contain sequences that are frequently methylated in plants, i.e., retrotransposons (Rabinowicz et al., 1999 Nature Genet., 23:305-308) and centromeric sequences (Moore et al., 1993 Genomics, 15:472-482) (see Table 2 and FIG. 7). Regardless, to construct a Cot library that best represents a particular Cot component, one should use a host strain with a genotype in which no insert sequence is excluded (or preferentially included) based on its methylation pattern.

### Example 7

DNA—DNA Hybridization Analysis

Southern blots containing S. bicolor and S. propinquum DNA were prepared and probed as described by Chittenden et al. (1994 Theor. Appl. Genet., 87:925-933). For simple determination of hybridization intensity, 15 clones from each Cot library were randomly selected as sources of probes. Clone inserts were preferentially amplified by PCR and labeled with ³²P-dCTP using nick translation. Each blot was hybridized with 1.8 ng/ml (=20 μCi/ml) of radiolabeled probe DNA in hybridization buffer for 16 hours at 65° C. Excess solution was drained from blots, and blots were given three successive 20 minute washes (65° C.) in 0.25×SSPE (aqueous 0.75 M NaCl, 50 mM NaH₂PO₄H₂O, 6.3 mM EDTA, pH 7.4) containing 0.25% SDS (1.0 L per wash with agitation). Membranes were blotted dry with paper towels and wrapped in plastic wrap. A Geiger-Muller counter was used to measure the relative amount of hybridization (cpm) of each probe to its corresponding blot.

The three Cot libraries differ in relative sequence iteration and composition in a manner reflecting the nature of the components from which they were derived, demonstrating that construction of repetition-based DNA libraries using Cot techniques is feasible. When Southern blots of sorghum genomic DNA were probed with randomly selected, radiolabeled Cot clone inserts, those blots hybridized with HRCot sequences exhibited a mean labeling intensity (cpm)>10 times that observed for MRCot-probed blots and >30 times that witnessed for SLCot-probed blots.

After sequence analysis, one of the S. bicolor/S. propinquum Southern blots was probed with radiolabeled insert from a Cot clone with substantial sequence identity to Retrosor-6 (HRCot3EO4). Hybridization conditions were identical to those described above. An autoradiogram of the blot was obtained using standard protocols.

High-density grids containing 18,432 double-spotted clones were prepared from the S. bicolor BAC library BTx623 and the S. propinquum library SP/YRL (Lin et al., 1999 Mol. Breeding, 5:511-520 as described by Choi and Wing, 1999 In Plant Molecular Biology Manual, S. Gelvin and R. Schilperoort, Eds. (The Netherlands: Kluwer Academic Publishers), pp. 1-32). For each BAC library, two identical BAC grids (i.e., two grids containing the same clones in the same order) were selected for analysis. One S. bicolor grid (SB1) and one S. propinquum grid (SPI) were each probed with part of the long terminal repeat (LTR) sequence (clone MRCot2BO4) of Retrosor-6 while the duplicate filters (SB2 & SP2) were probed with a sequence found in the central region of Retrosor-6 (clone HRCot3C12) (Choi and Wing, 1999 In Plant Molecular Biology Manual, S. Gelvin and R. Schilperoort, Eds. (The Netherlands: Kluwer Academic Publishers), pp. 1-32). Autoradiogram images were digitally captured using an Alpha Innotech (San Leandro, Calif.) Alphalmager 2200 image capture/analysis system. The two SB images were aligned, superimposed, and compared using Adobe Photoshop 6.0. SP images were likewise compared and analyzed.

S. bicolor and S. propinquum BAC grids probed with part of the Retrosor-6 LTR showed hybridization patterns nearly identical to those observed for duplicate blots probed with part of the internal region of the retroelement (FIGS. 8C-8D). Additionally, a Southern blot probed with a portion of the Retrosor-6 LTR exhibited a hybridization signal about twice that of a duplicate blot probed with an internal sequence of similar length, an observation indicating that there are roughly two copies of the LTR for each copy of the internal sequence. Based on the assumption that most copies of Retrosor-6 are similar to the kafirin cluster copy of the retroelement, densitometric analysis of BAC grids indicates that Retrosor-6 accounts for approximately six percent of the DNA in both sorghum species (Table 3). Without wishing to be bound by any particular theory, it is hypothesized that because S. bicolor and S. propinquum have similar genome sizes and possess roughly the same number of copies of Retrosor-6, the retroelement may have been introduced into a common ancestor of the two species rather than into the species separately. However, on the assumption that Retrosor-6 provides no selective advantage to the genome and hence can undergo mutation without influencing fitness, the preponderance of apparently intact copies of Retrosor-6 and the relatively high level of shared sequence identity between the LTRs of the kafirin cluster copy of Retrosor-6 (615/618 bp matches, S′=1171 bits) suggest that the retroelement may be fairly new to the Sorghum genus.

To estimate the Retrosor-6 copy number in the genomes of S. bicolor and S. propinquum, the Alphalmager Spot Densitometry application (Alphalmager 2200 v. 5.1) was used to analyze one section (i.e., one-sixth) of BAC grid SB1 and one section of grid SPI (see FIG. 8E). For each section, a region within the section containing no visible probe hybridization was selected and set as “background”. The “Integrated Density Value” (IDV=Σ (each pixel value—background)) for the entire section was then determined. Because BAC clones were double-spotted on the grids, the IDV of the section was divided by two to yield the “Section IDV.” Using a circular sampling tool with a fixed diameter slightly smaller than a clone, IDV readings were taken for fifty different clones ranging from the lowest detectable hybridization signal to the highest hybridization intensity (FIG. 8E). Clones were selected from all areas of a grid section. The mean density value of the five clones with the lowest IDVs (LowIDV) and the mean value of the five clones with the highest IDVs (HighlDV) were determined. For both S. bicolor and S. propinquum, comparison of the LowIDV and HighIDV indicate an approximately four-fold difference in clone hybridization intensity (see Table 3). It was assumed that the LowIDV represents clones with one copy of Retrosor-6, and therefore inferred that the HighIDV represents clones with four copies of Retrosor-6. To determine the mean number of clones per section, the SectionlDV was divided by the LowIDV. The resulting value was used to estimate the Retrosor-6 copy number per genome and the percentage of the genome composed of Retrosor-6 DNA as shown in Table 3.

Cot clone sequences were compared to a local database containing the sequences of roughly 2000 molecular markers on the sorghum molecular genetic map using standard BLAST (blastn) procedures (Altschul et al., 1997 Nucleic Acids Res., 25:3402). Three of the nine molecular markers recognized by Cot clones appeared to be rDNA sequences. The three “rDNA molecular markers” are found at essentially the same locus on S. bicolor linkage group C (Table 4). The 18S-5.8S-26S rDNA locus has been localized by fluorescence in situ hybridization to the longest S. bicolor mitotic metaphase chromosome (Sang and Liang, 2000 Genome, 43:918-922). Likewise, it has been recently demonstrated that the longest S. bicolor pachytene chromosome is the nucleolus organizer chromosome (Draye et al., 2001 Plant Physiol., 125:1325-1341). Consequently, it appears that S. bicolor mitotic metaphase chromosome 1, meiotic chromosome 1, and linkage group C are the same entity, making linkage group C the first sorghum linkage group to be assigned to a cytologically distinguishable chromosome. The chromosomal positions of Cot clones containing sequences with high sequence similarity to molecular genetic markers (S′≧76.28) are shown in Table 4.

### Example 8

Sorghum Cot Libraries are Used to Augment the Physical Maps of Genomes

Since the feasibility of Cot cloning has been demonstrated, the sorghum Cot libraries were then used to augment the information content of the rapidly-growing S. bicolor and S. propinquum physical maps (Draye et al., 2001 Plant Physiol. 125:1325-1341; Bowers et al., 2001 Plant Anim. Genome IX Conf., www.intl-pag.org/pag/9/abstracts/P5d 12.html). For example, Cot clones with homology to Retrosor-6 were used to determine the genetic and physical distribution of this element by evaluating co-localization of Retrosor-6 and genetically-mapped RFLPs on S. bicolor and S. propinquum BACs. This basic principle was used in the physical mapping of other repeat sequences, including those that were not previously sequenced/characterized. For example, the clone HRCot4C 10 exhibited the second highest level of hybridization to sorghum Southern blots of any of the randomly-selected clones (7000 cpm) but showed no significant homology to any of the GenBank or SUCEST Database sequences. Thus, screening of sorghum BAC libraries with HRCot4C10 enabled its frequency and distribution to be determined and permitted isolation and sequencing of the element(s) containing the HRCot4C1O sequence. Cot clone insert sequences with homology to characterized plant genes (see Table 2) were used to find sorghum homologues/orthologs in BAC clones and position these sequences on physical maps. Likewise, SLCot clone inserts that produced single-bands on Southern blots and showed homology to “non-repetitive” EST sequences were used to isolate, sequence, and map corresponding genes.

TABLE 1
Intra- and Interlibrary Comparison of Cot Clones With Significant Hits to ESTs.

| | HRCot | | MRCot | | SLCot | | HRCot + MRCot + SLCot | |
|---|---|---|---|---|---|---|---|---|
| | n = 253 | | n = 409 | | n = 499 | | n = 1161 | |
| Category | # | % | # | % | # | % | Total # | Total % |
| ECs^{a} | 136 | 53.8 | 190 | 46.5 | 152 | 30.5 | 478 | 41.2 |
| Repetitive ECs^{b} | 57 | 22.5 | 95 | 23.2 | 18 | 3.6 | 170 | 14.6 |
| ^{Nr-}ECs^{c} | 79 | 31.2 | 95 | 23.2 | 134 | 26.9 | 308 | 26.5 |
| | HRCot | | MRCot | | SLCot | | HRCot + MRCot + SLCot | |
| | n = 79 | | n = 95 | | n = 134 | | n = 308 | |
| ^{Nr-}EC Ψ^{d} | # | % | # | % | # | % | Total # | Total % |
| 0 | 24 | 30.4 | 23 | 24.2 | 118 | 88.1 | 165 | 53.6 |
| 1 | 8 | 10.1 | 13 | 13.7 | 4 | 3.0 | 25 | 8.1 |
| 2-5 | 17 | 21.5 | 16 | 16.8 | 3 | 2.2 | 36 | 11.7 |
| 6-10 | 24 | 30.4 | 14 | 14.7 | 0 | 0 | 38 | 12.3 |
| >10 | 6 | 7.6 | 29 | 30.5 | 9 | 6.7 | 44 | 14.3 |

^{a}ECs (EST-hit clones) are Cot clones with at least one significant hit to an EST sequence(s) in the GenBank EST and/or the SUCEST EST Databases.
^{b}Repetitive ECs are ECs with significant homology to known repeat sequences (e.g., rDNA, chloroplast DNA, retroelements, MITEs, etc.) in the GenBank Nr Database.
^{cNr-}ECs are ECs without significant homology to known repeat sequences in the GenBank Nr Database.
^{dNr-}ECs with a ι of “0” have no significant EST hits in common with other ECs, ^{Nr-}ECs with a ι of “1” share an EST hit(s) with one other EC, etc. At the extreme, six ^{Nr-}ECS in the HRCot library and 29 ^{Nr-}ECs in the MRCot library possess significant homology to a SUCEST EST recognized by a total of 48 ECs (the remaining 13 clones with hits to this EST sequence show primary GenBank Nr hits to rDNA or chloroplast DNA).

TABLE 2
BLAST-Based Categorization of HRCot, MRCot, and SLCot clones.

| | | HRCot | | MRCot | | SLCot | | |
|---|---|---|---|---|---|---|---|---|
| Blast categories^{a} | Subcategories^{a} | # | % | # | % | # | % | Ref./Acc.^{b} |
| No significant hit | | 90 | 35.6 | 199 | 48.7 | 339 | 67.9 | |
| Chloroplast DNA | | 0 | 0.0 | 41 | 10.0 | 5 | 1.0 | |
| Mitochondrial DNA | | 0 | 0.0 | 0 | 0.0 | 1 | 0.2 | |
| rDNA | 18S-5.8S-26SrDNA | 22 | 8.7 | 35 | 8.6 | 9 | 1.8 | Many refs. |
| | 55 rDNA | 2 | 0.8 | 0 | 0.0 | 0 | 0.0 | Many refs. |
| Centromeric repeat | Sorghum, pHind12 | 2 | 0.8 | 4 | 1.0 | 0 | 0.0 | Miller et al., 1998a |
| | Sorghum, pHind22 | 0 | 0.0 | 1 | 0.2 | 0 | 0.0 | Miller et al., 1998a |
| | Sorghum, pSau3A9 | 4 | 1.6 | 1 | 0.2 | 0 | 0.0 | Jiang et al., 1996 |
| | Sorghum, pSau3A10 | 3 | 1.2 | 0 | 0.0 | 0 | 0.0 | Miller et al., 1998b |
| | Sorghum, CEN38 | 1 | 0.4 | 0 | 0.0 | 0 | 0.0 | Zwick et al., 2000 |
| Retroelement^{c} | CACTA-type element/TNP-2 gene | 0 | 0.0 | 2 | 0.5 | 1 | 0.2 | He et al., 2000 |
| | Sorghum, Leviathan | 1 | 0.4 | 5 | 1.2 | 0 | 0.0 | U07815, U07816 |
| | Sorghum, Candystripe-1 | 2 | 0.8 | 0 | 0.0 | 0 | 0.0 | Chopra et al., 1999 |
| | Sorghum, Retrosor-2 | 1 | 0.4 | 1 | 0.2 | 3 | 0.6 | AF061282 |
| | Sorghum, Retrosor-6 | 34 | 13.4 | 7 | 1.7 | 0 | 0.0 | AF061282 |
| | Barley, cereba polyprotein pseudogene | 0 | 0.0 | 1 | 0.2 | 0 | 0.0 | Presting et al., 1998 |
| | Rice, gypsy-like integrase gene | 0 | 0.0 | 3 | 0.7 | 0 | 0.0 | AF244793 |
| | Maize, rev. tran./integr. pseudogene | 0 | 0.0 | 1 | 0.2 | 0 | 0.0 | AF030633 |
| | Sorghum, putative LTR | 1 | 0.4 | 0 | 0.0 | 0 | 0.0 | AF061282 |
| MITE^{c} | Putative MITE in sugarcane ubi9 gene | 0 | 0.0 | 3 | 0.7 | 0 | 0.0 | AF093505 |
| | Putative MITE in sorghum kafirin BAC | 0 | 0.0 | 0 | 0.0 | 1 | 0.2 | AF061282 |
| Other dispersed repeat^{c} | PREM-1-related repeat | 1 | 0.4 | 0 | 0.0 | 0 | 0.0 | Turcich et al., 1996 |
| | Sorghum HCSR-1 repeat | 0 | 0.0 | 0 | 0.0 | 1 | 0.2 | AF061282 |
| | Sorghum HCSR-7 repeat | 2 | 0.8 | 0 | 0.0 | 0 | 0.0 | AF061282 |
| | Johnsongrass XSR3 repeat^{d} | 0 | 0.0 | 1 | 0.2 | 0 | 0.0 | X54624 |
| | Johnsongrass XSR6 repeat^{d} | 1 | 0.4 | 0 | 0.0 | 0 | 0.0 | X54625 |
| | Sorghum, putative dispersed repeat | 1 | 0.4 | 2 | 0.5 | 0 | 0.0 | AF114171 |
| Characterized gene | Rice, bZIP DNA-binding factor | 0 | 0.0 | 0 | 0.0 | 1 | 0.2 | U04295 |
| | Rice, monosaccharide transporter 1 | 0 | 0.0 | 0 | 0.0 | 1 | 0.2 | AB052883 |
| | Barley, cp33Hv protein | 0 | 0.0 | 0 | 0.0 | 1 | 0.2 | AJ224325 |
| | Ice plant, protein kinase | 0 | 0.0 | 0 | 0.0 | 1 | 0.2 | Z30331 |
| | Maize, peroxidase gene | 0 | 0.0 | 0 | 0.0 | 1 | 0.2 | AJ401276 |
| | Sorghum, NADPH-dependent reductase | 0 | 0.0 | 0 | 0.0 | 1 | 0.2 | AF010283 |
| | Rice, OsNAC4 gene | 0 | 0.0 | 1 | 0.2 | 0 | 0.0 | AB028183 |
| | Canola, FCA gene | 0 | 0.0 | 1 | 0.2 | 0 | 0.0 | AJ237848 |
| Uncertain character^{e} | | 4 | 1.6 | 7 | 1.7 | 3 | 0.6 | |
| Repetitive EST | | 49 | 19.4 | 27 | 6.6 | 11 | 2.2 | |
| Ambiguous EST | | 11 | 4.3 | 11 | 2.7 | 23 | 4.6 | |
| Single/low copy EST | | 21 | 8.3 | 55 | 13.4 | 96 | 19.2 | |
| | TOTAL | 253 | 100 | 409 | 100 | 499 | 100 | |

^{a}Clones have been placed into thirteen “BLAST categories” according to FIG. 4. Some BLAST categories have been further divided into “subcategories”. For each Cot library, the number (#) and percentage (%) of clones in a BLAST category/subcategory are given.
^{b}A literary reference (Ref.) or GenBank Accession number (Acc.) is given for the sequence or sequences in a subcategory.
^{c}Cot clones categorized as “Retroelements”, “MITEs”, and “Other dispersed repeats” collectively constitute “Dispersed repeat sequences”.
^{d}Johnsongrass (Sorghum halepense Pers.), an extremely aggressive weed, appears to be an interspecific hybrid descendant (autoallotetraploid) of S. bicolor and S. propinquum (Paterson et at., 1995 Pers. Proc. Natl. Acad Sci. USA, 92: 6127-6131).
^{e}Genomic sequence of uncertain character

TABLE 3
Densitometric Analysis of BAC Grids Probed With Retrosor-6.

| | | | S. |
|---|---|---|---|
| Row | Description | S. bicolor | propinquum |
| A | Section IDV^{a} | 3,743,625 | 3,281,760 |
| B | Low IDV^{b} | 1230 | 1002 |
| C | High IDV^{b} | 4760 | 3912 |
| D | Range C ÷ B) | 3.87 | 3.90 |
| E | Copies of Retrosor-6 per section | 3043.6 | 3275.2 |
| | (A ÷ B) | | |
| F | BAC clones per section | 3072 | 3072 |
| G | Mean BAC insert size (bp)^{c} | 120,000 | 126,000 |
| H | BAC insert DNA per section in bp | 368,640,000 | 387,072,000 |
| | (F × G) | | |
| I | Genome size in bp^{d} | 760,000,000 | 772,000,000 |
| J | Fraction of genome in a section | 0.49 | 0.50 |
| | (H ÷ I) | | |
| K | Copies of Retrosor-6 per genome | 6211.4 | 6550.4 |
| | (E ÷ J) | | |
| L | Size of Retrosor-6 in bp | 7377 | 7377 |
| M | Bp of Retrosor-6 in genome | 45,821,498 | 48,322,301 |
| | (K × L) | | |
| N | Genome fraction in Retrosor-6 | 0.060 | 0.063 |
| | (M ÷ I) | | |

^{a}IDV or “Integrated Density Value” is the sum of all pixel values after background correction. Section IDV is the IDV of an entire grid section, i.e., one-sixth of a BAC grid (see FIG. 6E), divided by two to correct for double-spotting of clones.
^{b}Low IDV is the mean of the five colonies with the lowest IDVs while High IDV is the mean of the five colonies with the highest IDVs.
^{c}Mean insert size for S. bicolor BT × 623 BAC clones is from Begum et al., unpublished results (see www.clemson.genome.edu). Mean insert size for S. propinquum BAC clones is from Lin et al. (1999).
^{d}Genome size (1C) for S. bicolor is from Arumuganathan and Earle (1991). The genome size for S. propinquum is from K. Arumuganathan, personal communication as cited in Lin et al., 1999 Mol. Breeding, 5:511-520.

TABLE 4
Cot Clones Corresponding to Sorghum RFLP Markers.

| | Map | | |
|---|---|---|---|
| Marker^{a} | position(s)^{b} | Cot cones^{c} | BLAST category^{d} |
| AEST602 | C:46.2 | HRCot1C02 | rDNA |
| C0152 | C:46.2 | HRCot2E08, MRCot4G11, HRCot2E03 | rDNA |
| PRC0015 | C:46.0 | HRCot2A02 | rDNA |
| PRC1151 | A:59.3 | SLCot6G11 | Characterized gene^{e} |
| pSB0415 | H:40 | HRCot3F09, HRCot3F06, HRCot3B12, MRCot1F11 | No significant hits |
| pSB0986 | B:69.3 | SLCot1C09 | No significant hits |
| pSB1021 | C:91.6 | SLCot4H08 | No significant hits |
| pSB1524 | F:75.4 | MRCot1F03 | No significant hits |
| RZ014 | A:112.4 | SLCot4B11 | Single/low copy EST |

^{a}The molecular genetic marker to which one or more Cot clones shares significant homology (S′ ≧ 76.28).
^{b}Sorghum bicolor linkage group (traditionally represented by letters A through J) and map position (cM) are separated by a colon (e.g., B:33.5).
^{c}Cot clones producing a significant hit when BLASTed against all the marker sequences on the sorghum molecular genetic map
^{d}BLAST category is the group into which a Cot clone(s) was placed based upon the scheme shown in FIG. 4.
^{e}The characterized gene recognized by SLCot6G11 is the Zea may peroxidase-3 (pox3) gene (GenBank AJ401276).

We claim:

1. A method of producing a genomic DNA library of an organism, comprising:
a) performing renaturation kinetics based fractionation of the genomic DNA of the organism;
b) isolating more than one Cot component from the fractionated genomic DNA; and
c) preparing more than one Cot library from the more than one isolated Cot component, to thereby produce a genomic DNA library of an organism.

2. The method of claim 1, wherein preparing the Cot library comprises
a) ligating the isolated Cot component from the fractionated genomic DNA into a suitable vector; and
b) transforming a host cell with the vector comprising the isolated Cot component, to thereby prepare the Cot library.

3. The method of claim 1, wherein the isolated Cot component is selected from the group consisting of a fold-back DNA fraction, a highly repetitive DNA component, a moderately repetitive DNA component, and a single/low copy DNA component.

4. The method of claim 1, wherein the organism is a eukaryote.

5. The method of claim 1, wherein the organism is a fungus or a protist.

6. The method of claim 4, wherein the organism is a plant.

7. The method of claim 6, wherein the plant is a dicot.

8. The method of claim 6, wherein the plant is a monocot.

9. The method of claim 6, wherein the plant is selected from the group consisting of a conifer, a bryophyte, a fern, a homwort, a liverwort, a horsetail, a whisk-fern, a cycad, a gingko, and a gnetophyte.

10. The method of claim 4, wherein the organism is an animal.

11. The method of claim 10, wherein the animal is a vertebrate.

12. The method of claim 10, wherein the animal is an invertebrate.

13. The method of claim 1, wherein renaturation kinetics based fractionation is performed on genomic DNA fragmented by enzyme digestion, sonication, NaOH treatment, hydrodynamic shearing, or mechanical shearing.

14. The method of claim 1, wherein renaturation kinetics based fractionation is performed on genomic DNA having a length of between approximately 10 base pairs to approximately 10,000 base pairs.

15. The method of claim 1, wherein renaturation kinetics based fractionation is performed on genomic DNA having a length of between approximately 100 base pairs to approximately 1000 base pairs.

16. The method of claim 1, wherein renaturation kinetics based fractionation is performed on genomic DNA having a length of between approximately 300 base pairs to approximately 600 base pairs.

17. The method of claim 1, wherein at least one of the more than one isolated Cot components is further fractionated into more than one subcomponent prior to preparing a Cot library.

18. The method of claim 1, wherein the Cot library is prepared from ssDNA.

19. The method of claim 1, wherein the Cot library is prepared from dsDNA.

20. A method of determining the sequence complexity of the genomic DNA of an organism, comprising:
a) performing renaturation kinetics based fractionation of the genomic DNA of the organism;
b) isolating a Cot component from the fractionated genomic DNA;
c) preparing a Cot library from the isolated Cot component wherein the Cot library comprises a Cot clone; and
d) sequencing a Cot clone from the Cot library to a depth determined based on the kinetic complexity of the isolated Cot component from which the Cot library is prepared, thereby determining the sequence complexity of the genomic DNA of the organism.

21. The method of claim 20, wherein more than approximately 0.5% of the genome of the organism is sequenced.

22. The method of claim 20, wherein preparing the Cot library comprises
a) ligating the isolated Cot component from the fractionated genomic DNA into a suitable vector; and
b) transforming a host cell with the vector comprising the isolated Cot component, to thereby prepare the Cot library.

23. The method of claim 20, wherein the isolated Cot component is selected from the group consisting of a fold-back DNA fraction, a highly repetitive DNA component, a moderately repetitive DNA component, and a single/low copy DNA component.

24. The method of claim 20, wherein the organism is a eukaryote.

25. The method of claim 20, wherein the organism is a fungus or a protist.

26. The method of claim 24, wherein the organism is a plant.

27. The method of claim 26, wherein the plant is a dicot.

28. The method of claim 26, wherein the plant is a monocot.

29. The method of claim 26, wherein the plant is selected from the group consisting of a conifer, a bryophyte, a fern, a hornwort, a liverwort, a horsetail, a whisk-fern, a cycad, a gingko, and a gnetophyte

30. The method of claim 24, wherein the organism is an animal.

31. The method of claim 30, wherein the animal is a vertebrate.

32. The method of claim 30, wherein the animal is an invertebrate.

33. The method of claim 20, wherein renaturation kinetics based fractionation is performed on genomic DNA fragmented by enzyme digestion, sonication, NaOH treatment, hydrodynamic shearing, or mechanical shearing.

34. The method of claim 20, wherein renaturation kinetics based fractionation is performed on genomic DNA having a length of between approximately 10 base pairs to approximately 10,000 base pairs.

35. The method of claim 20, wherein renaturation kinetics based fractionation is performed on genomic DNA having a length of between approximately 100 base pairs to approximately 1000 base pairs.

36. The method of claim 20, wherein renaturation kinetics based fractionation is performed on genomic DNA having a length of between approximately 300 base pairs to approximately 600 base pairs.

37. The method of claim 20, wherein the isolated Cot component is further fractionated into more than one subcomponent prior to preparing a Cot library.

38. The method of claim 20, wherein the Cot library is prepared from ssDNA.

39. The method of claim 20, wherein the Cot library is prepared from dsDNA.

40. A method of cloning the single/low copy genomic DNA of an organism, comprising:
a) performing renaturation kinetics based fractionation of the genomic DNA of the organism;
b) isolating a single/low copy Cot component comprising a DNA sequence from the fractionated genomic DNA, wherein the DNA sequence is present from approximately 1 to approximately 10 copies per haploid genome; and
c) preparing a Cot library from the single/low copy Cot component wherein the Cot library comprises a Cot clone, thereby cloning the single/low copy genomic DNA of the organism.

41. The method of claim 40, further comprising sequencing a Cot clone from the Cot library to a depth determined based on the kinetic complexity of the Cot component.

42. The method of claim 41, wherein more than approximately 0.5% of the genomic DNA of the organism is sequenced.

43. A method of cloning the moderately repetitive genomic DNA of an organism, comprising:
a) performing renaturation kinetics based fractionation of the genomic DNA of the organism;
b) isolating a moderately repetitive Cot component comprising a DNA sequence from the fractionated genomic DNA, wherein the DNA sequence is present at more than approximately 10 copies per haploid genome; and
c) preparing a Cot library from the moderately repetitive Cot component wherein the Cot library comprises a Cot clone, thereby cloning the moderately repetitive genomic DNA of the organism.

44. The method of claim 43, further comprising sequencing a Cot clone from the Cot library to a depth determined based on the kinetic complexity of the Cot component.

45. The method of claim 44, wherein more than approximately 0.5% of the genomic DNA of the organism is sequenced.

46. A method of cloning the highly repetitive genomic DNA of an organism, comprising:
a) performing renaturation kinetics based fractionation of the genomic DNA of the organism;
b) isolating a highly repetitive Cot component comprising a DNA sequence from the fractionated genomic DNA, wherein the DNA sequence is present at more than approximately 5000 copies per haploid genome; and
c) preparing a Cot library from the highly repetitive Cot component wherein the Cot library comprises a Cot clone, thereby cloning the highly repetitive DNA of the organism.

47. The method of claim 46, further comprising sequencing a Cot clone from the Cot library to a depth determined based on the kinetic complexity of the Cot component.

48. The method of claim 47, wherein more than approximately 0.5% of the genomic DNA of the organism is sequenced.

49. A method of determining the sequence of the genomic DNA of an organism, comprising:
a) performing renaturation kinetics based fractionation of the genomic DNA of the organism;
b) isolating a Cot component from the fractionated genomic DNA; and
c) sequencing the Cot component to a depth determined based on the kinetic complexity of the isolated Cot component, thereby determining the sequence of the genomic DNA of the organism.

50. The method of claim 49, wherein performing renaturation kinetics based fractionation of the genomic DNA comprises the preparation of a Cot curve.

51. The method of claim 50, wherein the Cot curve comprises one Cot component.

52. The method of claim 50, wherein the Cot curve comprises two Cot components.

53. The method of claim 50, wherein the Cot curve comprises three Cot components.

54. The method of claim 49, wherein the isolated Cot component is selected from the group consisting of a fold-back DNA fraction, a highly repetitive DNA component, a moderately repetitive DNA component, and a single/low copy DNA component.

55. The method of claim 49, wherein the organism is a eukaryote.

56. The method of claim 49, wherein the organism is a fungus or a protist.

57. The method of claim 55, wherein the organism is a plant.

58. The method of claim 55, wherein the organism is an animal.

59. The method of claim 58, wherein the animal is a vertebrate.

60. The method of claim 58, wherein the animal is an invertebrate.

61. The method of claim 49, wherein renaturation kinetics based fractionation is performed on genomic DNA fragmented by enzyme digestion, sonication, NaOH treatment, hydrodynamic shearing, or mechanical shearing.

62. The method of claim 49, wherein renaturation kinetics based fractionation is performed on genomic DNA having a length of between approximately 300 base pairs to approximately 600 base pairs.

63. The method of claim 49, wherein the isolated Cot component is further fractionated into more than one subcomponent prior to sequencing.

64. A kit for use in nucleic acid sequencing, comprising a Cot library, wherein the Cot library is prepared from a Cot component isolated from the genomic DNA of an organism by renaturation kinetics based fractionation of the genomic DNA, and wherein the Cot component is selected from the group consisting of a single/low copy Cot component comprising a DNA sequence present from approximately 1 to approximately 10 copies per haploid genome, a moderately repetitive Cot component comprising a DNA sequence present at more than approximately 10 copies per haploid genome, and a highly repetitive Cot component comprising a DNA sequence present at more than approximately 5000 copies per haploid genome.

65. The kit of claim 64, wherein the organism is a eukaryote.

66. The kit of claim 64, wherein the organism is a fungus or a protist.

67. The kit of claim 65, wherein the organism is a plant.

68. The kit of claim 67, wherein the plant is a dicot.

69. The kit of claim 67, wherein the plant is a monocot.

70. The kit of claim 67, wherein the plant is selected from the group consisting of a conifer, a bryophyte, a fern, a hornwort, a liverwort, a horsetail, a whisk-fern, a cycad, a gingko, and a gnetophyte.

71. The kit of claim 65, wherein the organism is an animal.

72. The method of claim 71, wherein the animal is a vertebrate.

73. The method of claim 71, wherein the animal is an invertebrate.

74. The kit of claim 64, wherein renaturation kinetics based fractionation is performed on genomic DNA fragmented by enzyme digestion, sonication, NaOH treatment, hydrodynamic shearing, or mechanical shearing.

75. The kit of claim 64, wherein renaturation kinetics based fractionation is performed on genomic DNA having a length of between approximately 10 base pairs to approximately 10,000 base pairs.

76. The kit of claim 64, wherein renaturation kinetics based fractionation is performed on genomic DNA having a length of between approximately 100 base pairs to approximately 1000 base pairs.

77. The kit of claim 64, wherein renaturation kinetics based fractionation is performed on genomic DNA having a length of between approximately 300 base pairs to approximately 600 base pairs.

78. The kit of claim 64, wherein the isolated Cot component is further fractionated into more than one subcomponent prior to preparing the Cot library.

79. The kit of claim 64, wherein the Cot library is prepared from ssDNA.

80. The kit of claim 64, wherein the Cot library is prepared from dsDNA.
